(19) United States
(12) Patent Application Publication
(10) Pub. No.: US 2020/0129760 A1
(43) Pub. Date: Apr. 30, 2020

(54) WIRELESS AUDIO DEVICE

(71) Applicant: Otoharmonics Corporation, Portland, OR (US)

(72) Inventors: Michael BAKER, Portland, OR (US); Leonardo MARTINEZ HORNAK, Ciudad de la Costa (UY); Leonardo Raul CECILA DELGADO, Montevideo (UY)

(21) Appl. No.: 16/496,939

(22) PCT Filed: Mar. 21, 2018
(86) PCT No.: PCT/US18/23639

§ 371 Date: Sep. 23, 2019

Related U.S. Application Data
(60) Provisional application No. 62/474,589, filed on Mar. 21, 2017.

(51) Int. Cl. A61N 1/36; A61B 5/12; H04R 25/00; A61B 5/00; H04R 1/10; A61B 5/0205

(52) U.S. Cl. CPC A61N 1/361; A61B 5/128; H04R 25/75; A61B 5/6803; A61B 5/02055; A61B 5/6817; A61B 5/4812; H04R 1/1016; A61B 5/7415

(57) ABSTRACT

Methods and systems are provided for a sound device for making or treatment of tinnitus. In one example, a method includes determining a current sleep cycle and administering a therapy sound based on the current sleep cycle.

### CROSS REFERENCE TO RELATED APPLICATION

The present application claims priority to U.S. Provisional Application No. 62/474,589, entitled “Wireless Audio Device”, and filed on Mar. 21, 2017. The entire contents of the above-listed application are hereby incorporated by reference for all purposes.

### FIELD

The present description relates generally to a sound device for making or treatment of tinnitus.

### BACKGROUND/SUMMARY

Tinnitus is the sensation of hearing sounds when there are no external sounds present and can be loud enough to attenuate the perception of outside sounds. Tinnitus may be caused by inner ear cell damage resulting from injury, age-related hearing loss, and exposure to loud noises. The tinnitus sound perceived by the affected patient may be heard in one or both ears and also may include ringing, buzzing, clicking, and/or hissing.

Some methods of tinnitus treatment and/or therapy include producing a sound in order to mask the tinnitus of the patient. One example is shown by U.S. Pat. No. 7,850,596 where the masking treatment involves a pre-determined algorithm that modifies a sound similar to a patient's tinnitus sound.

However, the inventors herein have recognized that the therapy may be advantageously applied during selected sleep cycles, and/or that the selection of the type of sounds generated relative to a matched sound may be adjusted responsive to a current point in a sleep cycle of the user.

In one example, the issues described above may be addressed by a method comprising gathering biometric data from one or more sensors, such as located in an earbud of a wireless audio device, wherein the earbuds are pressed into a patient's ear, and where the wireless audio device is configured to analyze the biometric data and determine a current sleep cycle and administer a tinnitus sound therapy based on the current sleep cycle. In this way, tinnitus making or treatment sounds may be modified in real-time based on biometric data gathered.

As one example, the biometric data includes heat rate, respiration, body temperature, and blood pressure. The wireless audio device may be configured to determine transitions between sleep cycles while the patient is sleeping and play tinnitus making or treatment sounds based on a determined sleep cycle. The tinnitus making or treatment sounds may be adjusted based on one or more of biometric data gathered following administration of the tinnitus making or treatment sounds and progress through a current sleep cycle. As an example, if the biometric data changes in an undesirable direction in response to the tinnitus making or treatment sounds, then the sounds may be adjusted. For example, the type of sounds may be changed and/or a volume of the sounds may be adjusted. Additionally or alternatively, the tinnitus making or treatment sounds may be adjusted as a sleep cycle approaches a transitional period (e.g., transitioning from a current sleep cycle to a subsequent different cycle). During the transitional period, a volume of the tinnitus making or treatment sounds may be phased. For example, the volume may slowly increase at the start of a sleep cycle and slowly decrease near the end of the sleep cycle.

In another example, a method of applying sounds to a user's ear may be adjusted responsive to biometric data of the user during a sleep cycle. The selected sound may be adjusted responsive to transitions in the sleep cycle, as sensed by the biometric data in real-time. Further, the volume of the sound may be adjusted based on the sleep cycle, including phase-in and phase-out volume timing.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIGS. 1A-1D show schematic diagrams of example devices for a tinnitus therapy including a patient's device.

FIG. 2 shows the patient's device.

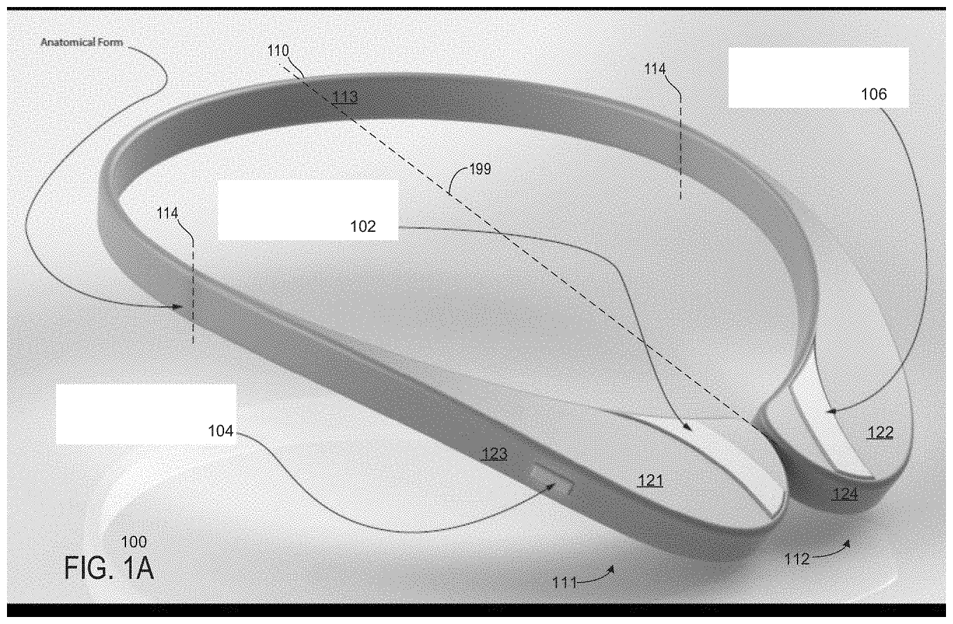

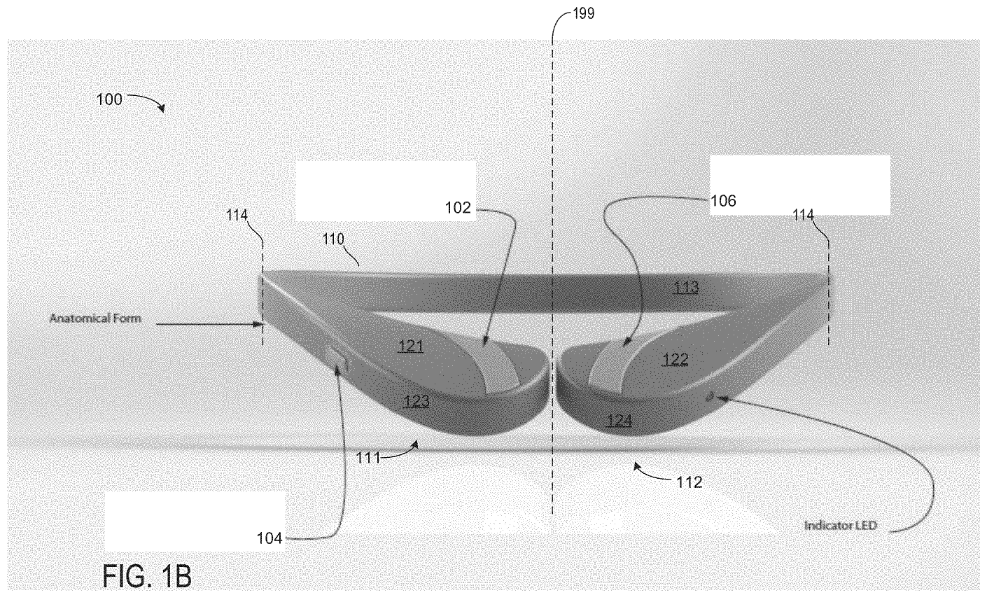

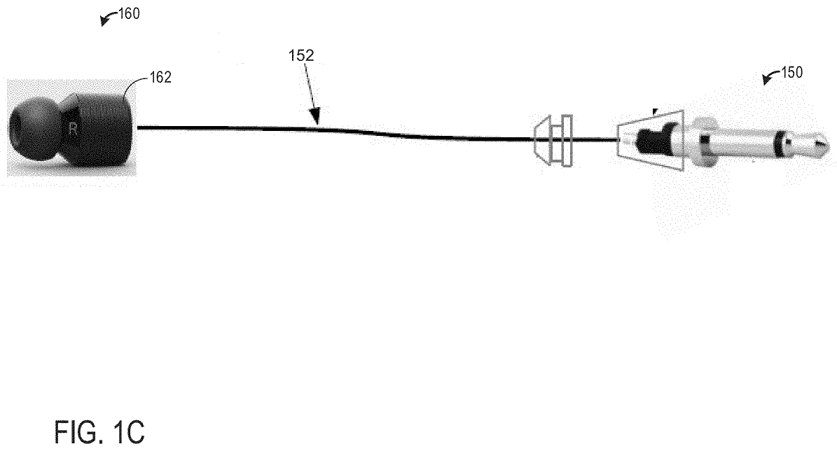

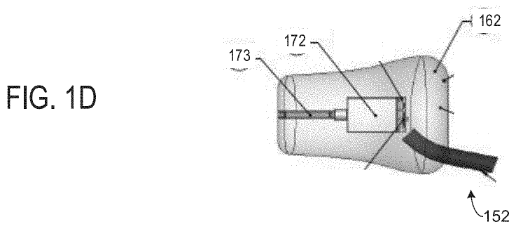

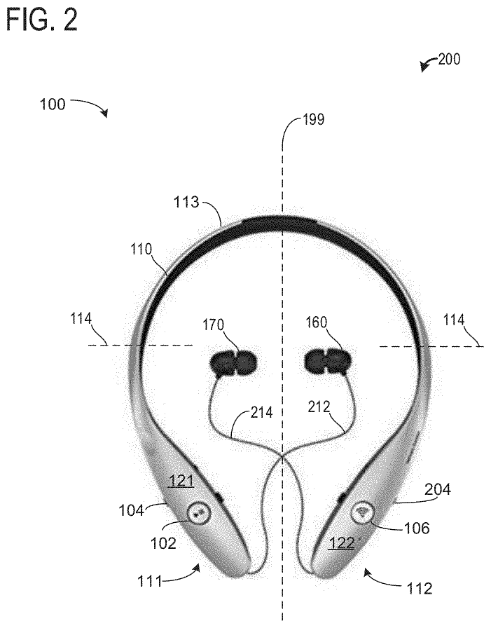

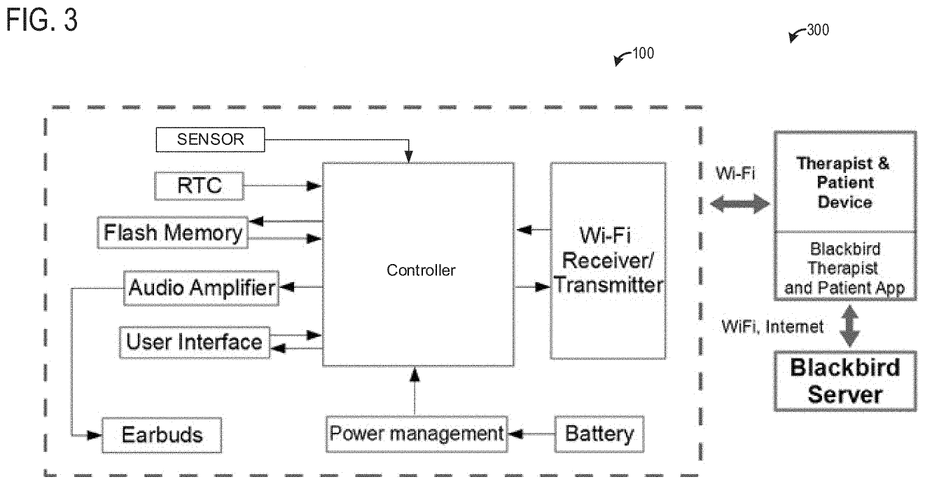

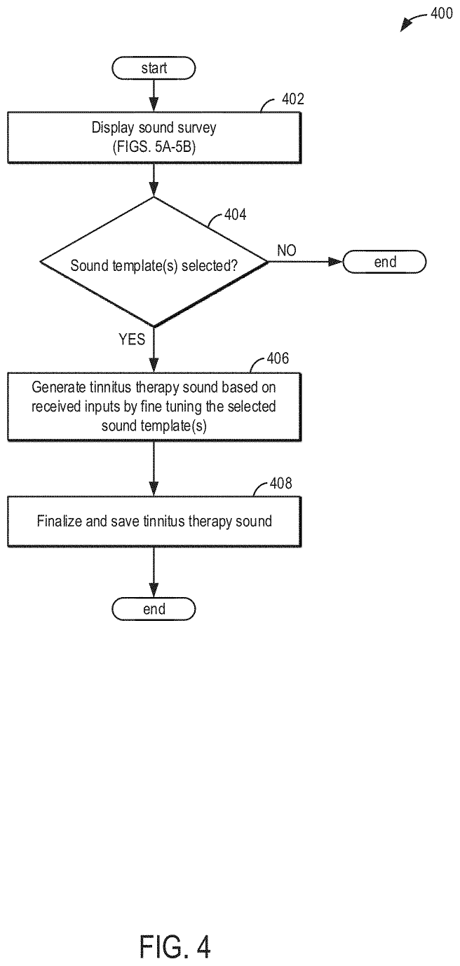

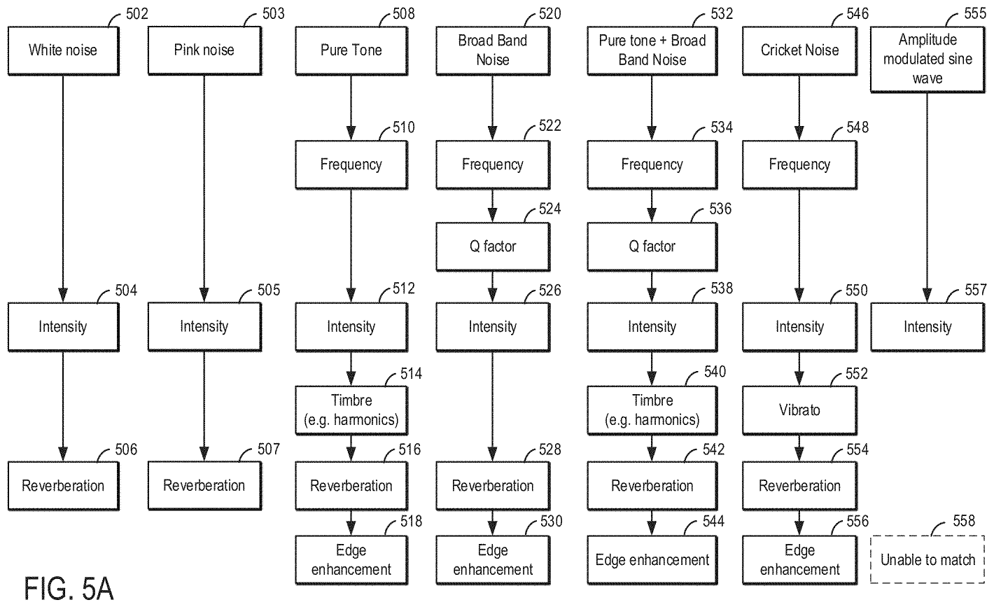

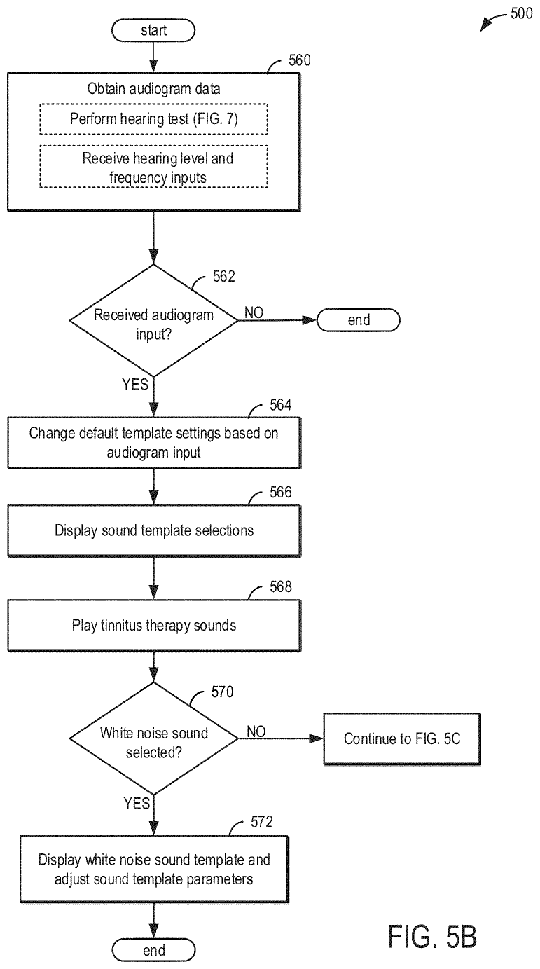

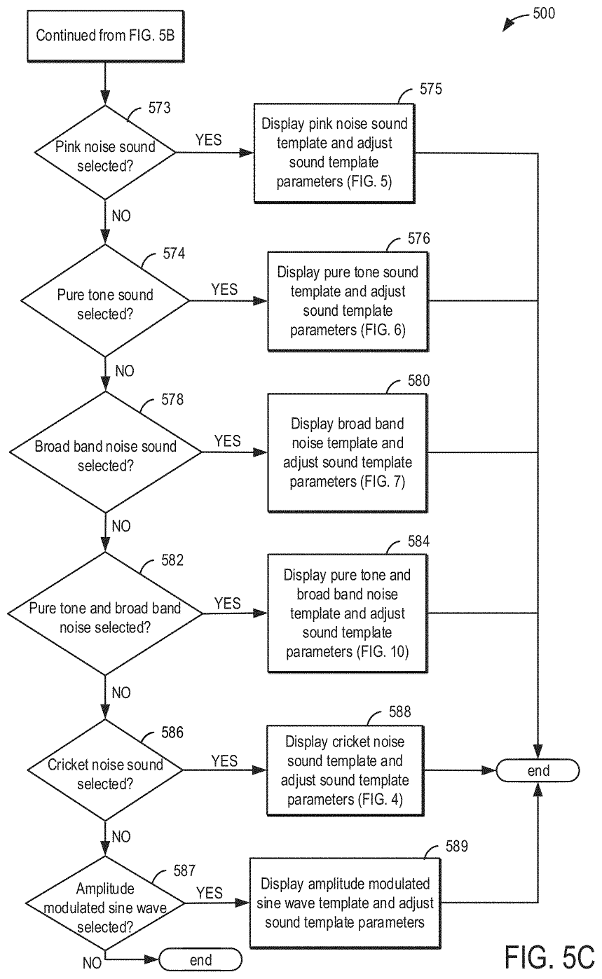

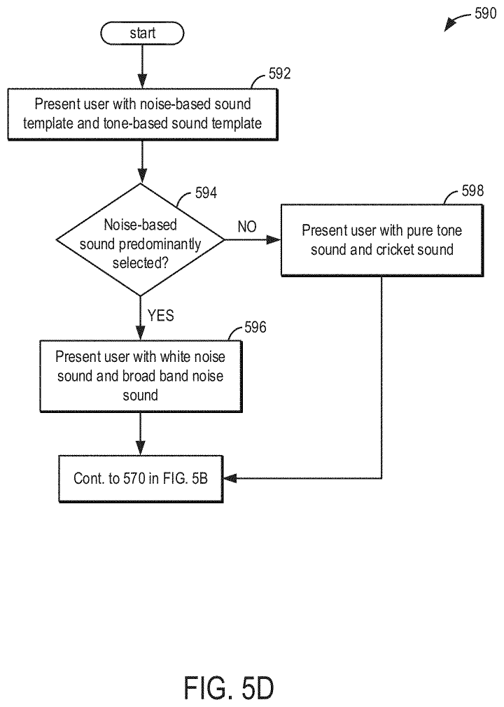

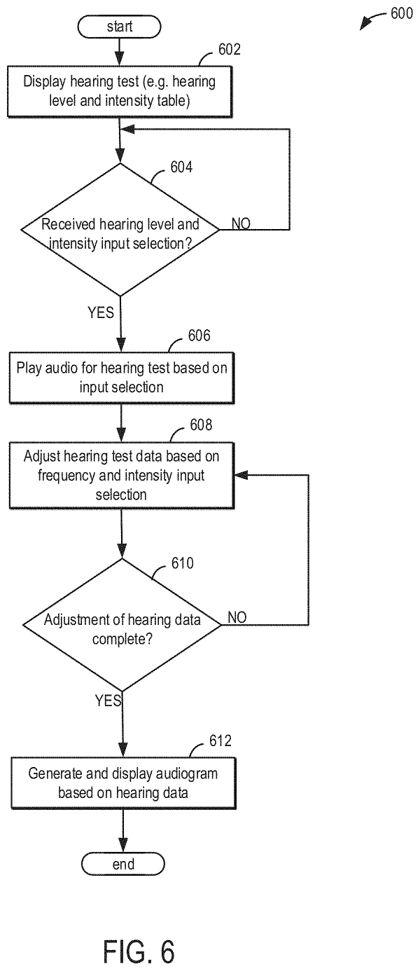

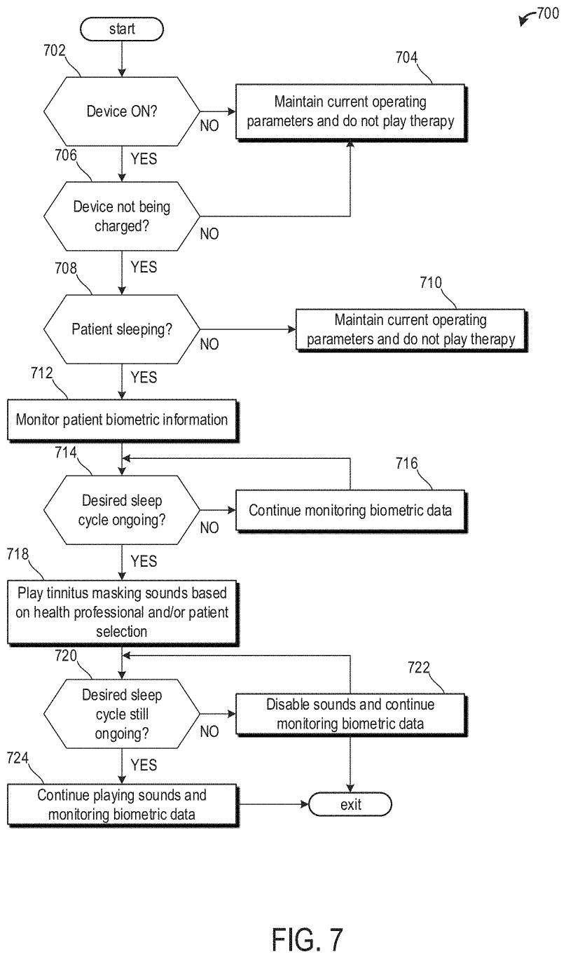

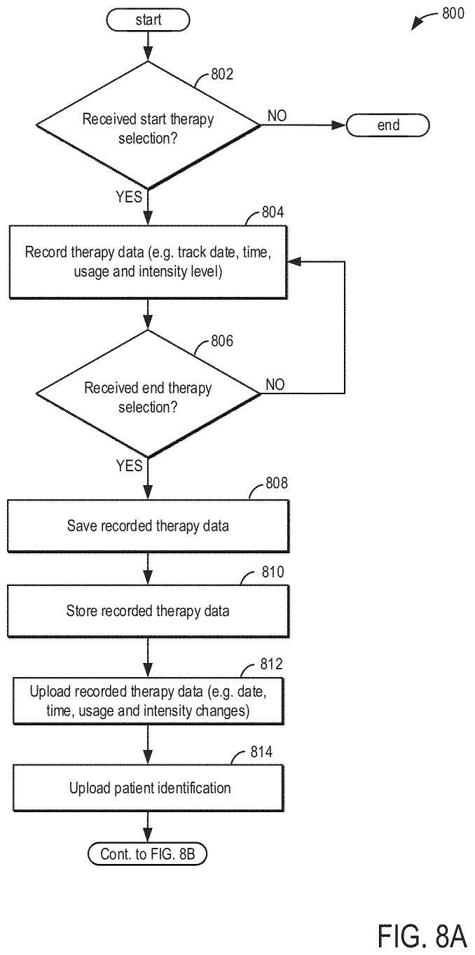

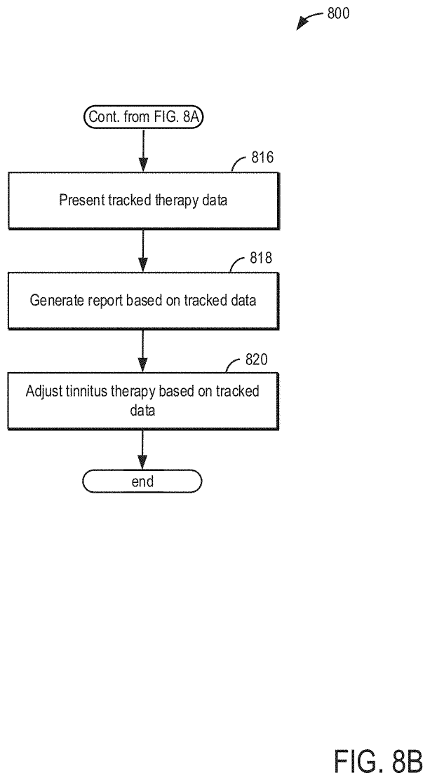

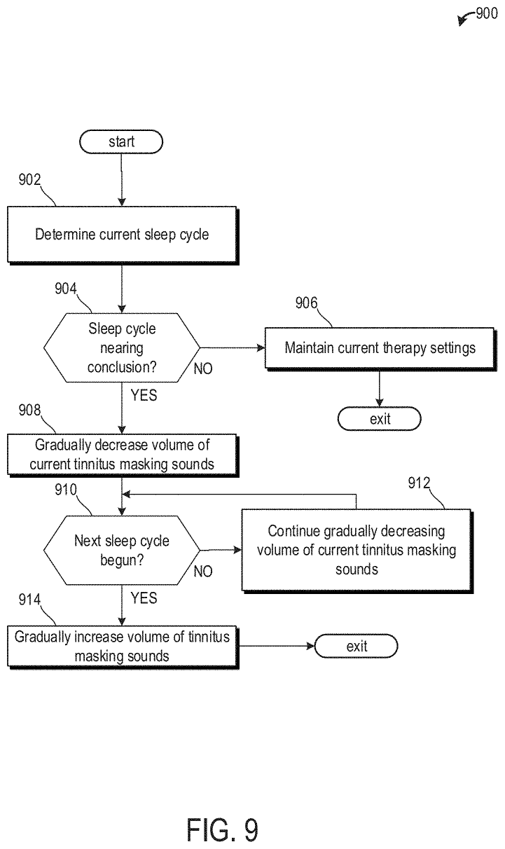

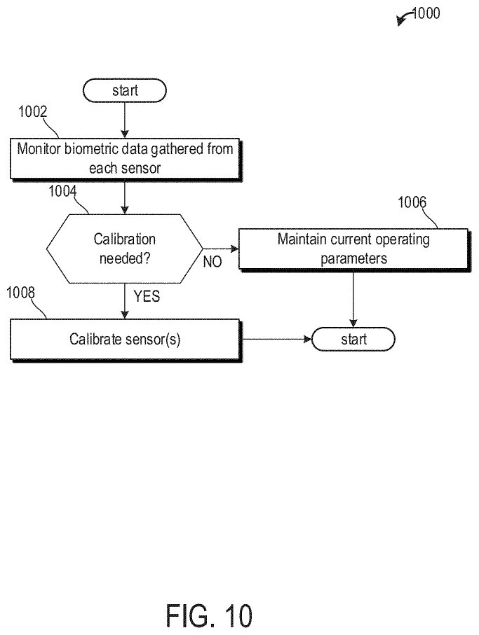

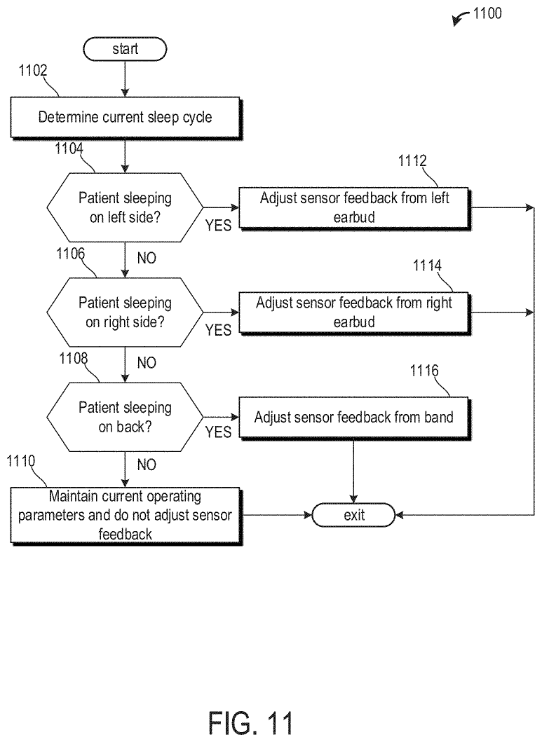

FIGS. 1A-1D and FIG. 2 are shown approximately to scale. Although, other relative dimensions may be used without departing from the scope of the present disclosure.

FIG. 3 shows a high-level chart depicting hardware components of the wireless audio device and its relation to an auxiliary device of a patient and/or healthcare provider.

FIG. 4 shows a method for monitoring biometric data of the patient.

FIGS. 5A, 5B, 5C, and 5D show example methods for generating a sound survey.

FIG. 6 shows an example method for generating an audiogram.

FIG. 7 shows a method for administering tinnitus therapy sounds to a patient during the patient's sleep.

FIGS. 8A and 8B show an example method for tracking patient data.

FIG. 9 shows a method for adjusting tinnitus making or treatment sounds near the beginning and/or conclusion of a current sleep cycle.

FIG. 10 shows a method for calibrating one or more sensors of the tinnitus making or treatment device.

FIG. 11 shows a method for determining a sleeping position of the patient.

### DETAILED DESCRIPTION

Methods and systems are provided for tinnitus therapy generation, tracking, and reviewing. In another example, the methods and systems may be adapted for other audio therapies or neurological disorders and treatments. In one embodiment, tinnitus therapy for the treatment of tinnitus may include therapy sessions and tracking of the therapy sessions generated and carried out on a patient's device, such as the patient's device shown in FIGS. 1A-1D. FIG. 2 shows an embodiment of the patient's device. The device comprises sensors configured to measure one or more biometric data values. This may include but is not limited to one or more of blood pressure (BP), heart rate (HR), respiration, and body temperature. FIG. 3 shows a high-level figure illustrating components located within the wireless audio device. FIG. 4 shows a method for implementing a sound survey. FIGS. 5A, 5B, 5C, and 5D show a method for sound template parameters. FIG. 6 shows a method for displaying an audiogram. FIG. 7 shows a method for determining a patient's sleeping cycle by monitoring the patient's biometric data and formulating a tinnitus therapy based on the determined sleeping cycle. FIGS. 8A and 8B shows a method for uploading patient data. FIG. 9 shows a method for adjusting tinnitus making or treatment sounds near the beginning and/or conclusion of a current sleep cycle. FIG. 10 shows a method for calibrating one or more sensors of the tinnitus making or treatment device. FIG. 11 shows a method for determining a sleeping position of the patient.

The tinnitus therapy may include a tinnitus therapy sound generated via the healthcare professional's device. The tinnitus therapy sound may be based on and include one or more types of sounds. For example, different types of sounds such as white noise, pink noise, pure tone, broad band noise, and cricket noise may be included in the tinnitus therapy sound. Specific tinnitus therapy sounds, or sound templates, may be pre-determined and include a white noise sound, a pink noise sound, a pure tone sound, a broad band noise sound, a cricket noise sound, an amplitude modulated sine wave, and/or a combine tone sound. A user may be presented with one or more of the above tinnitus therapy sound templates via the healthcare professional's device. Using a plurality of user interfaces of the healthcare professional's device, a user may select and modify one or more tinnitus therapy sound templates in order to generate a tinnitus therapy sound similar to the user's or patient's perceived tinnitus. However, the modifications do not include adding further amplitude of frequency modulation to the templates. In one example, a user may include a medical provider such as a physician, nurse, technician, audiologist, or other medical personnel. In another example, the user may include a patient.

FIGS. 1A-1D and FIG. 2 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example. It will be appreciated that one or more components referred to as being “substantially similar and/or identical” differ from one another according to manufacturing tolerances (e.g., within 1-5% deviation).

Turning now to FIG. 1A, it shows a wireless sound device 100 for a tinnitus therapy that may be used as a healthcare professional's device and/or a patient's device. In one example, the device 100 may be operated by a medical provider including, but not limited to, physicians, audiologists, nurses, and/or technicians. In another examples, the device 100 may be operated by a patient. Thus, the user of the healthcare professional's device may be one or more of a patient or a medical provider. Further, the user of the patient's device may be the patient.

The band 110 is U-shaped which may be located around a user's neck from a back of the neck. Specifically, the band 110 may rest on a patient's shoulders around their neck. Band 110 may extend around 30 to 70% of a circumference of the patient's neck. In one example, the band 110 is formed of a bendable material. The bendable material may include one or more of rubber and silicon. In this way, the band 110 may bend and/or twist without snapping and/or cracking.

The band 110 comprises two extreme ends, including right extreme end 111 and left extreme end 112. The extreme ends are rigid relative to a most bend portion of the band 110 (e.g., portion 113 located between the right 111 and left 112 extreme ends). It will be appreciated that the right extreme end 111 is located on a left side of the figure and the left extreme end 112 is located on a right side of the figure. For example, an elasticity of the band 110 is low between dashed lines 114 and the extreme ends compared to portion 113 located between the dashed lines 114. In this way, a user may move the extreme ends of the band 110 apart (e.g., further away from an axis 199) without bending the extreme ends. Said another way, the band 110 may bend at locations similar to the locations of the dashed lines 114 as a user fits the device 100 to a patient's head.

The device 100 expands in width from lines 114 to right 111 and left 112 extreme ends. As such, the device 100 comprises its greatest width at the right 111 and left 112 extreme ends. As shown, the right 111 and left 112 extreme ends are substantially identical. Additionally, the device 100 is substantially uniform along portion 113 of the band 110. In this way, the device 100 is symmetric along the axis 199.

A first button 102 for playing and pausing sounds emitting from speakers of the device 100 is arranged along a top surface 121 the right extreme end 111. The first button 102 is trapezoidal with contoured sides and edges. Said another way, the first button 102 is an asymmetric trapezoid with rounded edges and sides. The first button 102 follows a curvature of the right extreme end 111 along its short extreme ends.

The first button 102 is configured to play or pause sounds emitting from the device 100 when the first button is depressed for less than a first threshold duration (e.g., 2 seconds) while the device 100 is on. If the first button is depressed while the device is on for greater than the first threshold duration and less than a second threshold duration (e.g., four seconds), then the device 100 may be turned off. If the first button is depressed for greater than or equal to the second threshold duration while the device is on, then the device 100 may be restored to factory settings, wherein data saved on the device is erased. In one example, if the device 100 is off and the first button 102 is depressed, then the device 100 is turned on, regardless of a duration of time the first button is depressed. In one example, the device 100 may comprise audio prompts for alerting a user of a mode entered. For example, if the first button 102 is depressed for an amount of time between the second and third threshold durations while the device is on, then an audio recording may say, “device powering down” or “device off.”

A second button 104 for adjusting a volume of sounds emitting from speakers of the device 100 is arranged along a side surface 123 between the right extreme end 111 and the line 114. In one example, the second button 104 is biased toward the right extreme end 111 such that it is closer to the first button 102 than the right dashed line of the dashed lines 114. The second button 104 is slidable in one example. Sliding the second button 104 away from the right extreme end 111 may result in decreasing the volume of the device 100. Thus, sliding the button 104 toward the right extreme end 111 may result in increasing the volume of the device 100. In one example, the second button 104 may comprise an LED backlight configured to adjust brightness based on a volume selected. For example, as the volume of the device 100 increases based on an actuation of the second button 104, the backlight may increase in intensity (e.g., get brighter). If the volume of the device 100 decreases based on an actuation of the second button 104, the backlight may decrease in intensity. It will be appreciated that the backlight may decrease in intensity as the volume increases without departing from the scope of the present disclosure.

Additionally or alternatively, the second button 104 may comprise two separate buttons located at its extreme ends. For example, a portion of the second button 104 may comprise a volume increase button at an extreme end proximal to the right extreme end 111 and a volume decrease button at an extreme end distal to the right extreme end 111. As such, the second button 104 is fixed and its extreme ends may be depressed to adjust a volume of the device 100.

In some examples, additionally or alternatively, the second button 104 for adjusting volume of the device 100 may be arranged at both the right 111 and left 112 extreme ends. In this way, the device 100 may comprise a first volume button located on the right extreme end 111 and a second volume button located on the left extreme end 112. The first volume button may adjust a volume output of a speaker arranged in the right extreme end 111. The second volume button may adjust a volume output of a speaker arranged in the left extreme end 112. In this way, the first and second volume buttons provide a user with independent right and left volume controls, respectively.

A third button 106 for wirelessly connecting the device 100 to an auxiliary device is arranged on a top surface 122 of the left extreme end 112. A shape of the third button 106 is substantially identical to a shape of the first button 102. Thus, the third button 106 follows a curvature of the left extreme end 112 formed via a shape of a side surface 124. It will be appreciated that the third button 106 and first button 102 may be different shapes without departing from the scope of the present disclosure. For example, the first 102 and third 106 buttons may be circular, rectangular, pentagonal, etc. Additionally or alternatively, the first 102 and third 106 button may be different shapes than one another (e.g., the first button 102 is square and the third button 106 is rectangular).

The device 100, whether it be a healthcare professional's device or a patient's device, are physical, non-transitory devices configured to hold data and/or instructions executable by a logic subsystem. The logic subsystem may include individual components that are distributed throughout two or more devices, which may be remotely located and/or configured for coordinated processing. One or more aspects of the logic subsystem may be virtualized and executed by remotely accessible networked computing devices. The device 100 may display information to the user via connecting to an auxiliary device. In one example, the connection between the device 100 and the auxiliary device is via Bluetooth.

Bluetooth is a near field communication technical standard for connecting two hand-carry devices (e.g., mobile terminals, notebooks, earphones and headphones) to exchange information with each other and it is used when low power wireless connection is needed in an ultra-short range of 10-20 meters. Bluetooth uses 2400-2483.5 MHz which is ISM (Industrial Scientific and Medical) frequency band.

To block interference of other systems using upper and lower frequencies, Bluetooth uses total 79 channels of 2402-2480 MHz except a range of 2 MHz higher than 2400 MHz and 3.5 MHz lower than 2483.5 MHz. ISM is a frequency band assigned for industrial, scientific, and medical use and it is used in a personal wireless device which can emit low power electric waves, without permission to use electric waves. Amateur radio, wireless LAN and Bluetooth uses the ISM band.

Additionally or alternatively, the connection between the device 100 and the auxiliary device 100 may be via Wi-Fi. Wi-Fi is an example of far field communication and may allow the device 100 to connect to an auxiliary device distal to the device 100. As an example, the device 100 may be located in a patient's home while being connecting to a computing device located in a healthcare professional's office. In this way, the device 100 may relay information from the device 100 to an auxiliary device proximal or distal to a user.

At any rate, the device 100 may pair with the auxiliary device in response to a user depressing the third button 106. Quickly depressing the third button 106 (e.g., for less than a first threshold duration) signals to the device 100 to search for an auxiliary device. Alternatively, quickly depressing the third button 106 signals to the device 100 to connect to a Wi-Fi network, wherein an auxiliary device may access information gathered by the device 100. If the third button 106 is depressed for a duration of time greater than or equal to the first threshold duration and less than the second threshold duration, then the device 100 may search for any known networks. If the third button is depressed for a duration of time greater than or equal to the third threshold duration, then the user may set network parameters of access points for the device to connect to via Wi-Fi. In one example, an audio recording stored on the device may notify the user of each of the above modes. As an example, if the third button is depressed for greater than the third threshold duration, then a recording may say, “Enter Wi-Fi settings.” In this way, the third button 106 may be a dual functioning button, wherein a duration of a depression of the button adjusts its functionality.

In some examples, the first button 102 is configured to deactivate (e.g., turn off) the device 100. For example, if the first button is depressed for longer than the threshold time, then the device 100 may be turned off. However, if the device 100 is depressed for less than the threshold time, then audio from the device 100 begins to play or pauses.

Turning now to FIG. 1B, a face-on view of the device 100 is shown. An indicator light 108 is exposed in the face-on view located on the side surface 124 of the device 100. In one example, the indicator light 108 is a binary light, wherein the indicator light 108 is off when the device 100 is off and the indicator light 108 is on when the device 100 is on. Additionally or alternatively, the indicator light 108 may be configured to flash and/or pulse in response to a battery power of the device 100. For example, if the device 100 has less than a threshold state of charge (e.g., SOC corresponding to one hour or less of play time), then the indicator light 108 may begin to pulse.

Although not depicted, a right side of the device (e.g., the portion of the device 100 to a left of the axis 199) may further comprise a charging port, audio jack, indicator light, and RGB LED indicator. Additionally, a left side of the device may further comprise a battery. It will be appreciated that the left side may also include one or more of the charging port, audio jack, indicator light, and RGB LED indicator without departing from the scope of the present disclosure.

Turning now to FIG. 1C, it shows internal componentry 150 located inside of the right extreme end of the device (e.g., right extreme end 111 of the device 100). It will be appreciated that internal componentry 150 located within the right extreme end may also be located within the left extreme end of the device without departing from the scope of the present disclosure. The internal componentry is physically and electrically coupled to a right earbud 160 via a transducer cable 152. The earbud 160 comprises a mold 162, which may be selected based on specific measured geometries of a patient's ear. Thus, the mold 162 is customizable for each patient. In one example, the mold 162 may be easily removed and/or installed such that the wireless audio device may be interchangeably used among different patients.

As shown, the earbud 160 comprises an ingot (e.g., imprinted letter R) and/or some other form of marking (e.g., bump, etching, etc.) indicating the earbud 160 is the right earbud 160. In this way, the mold 162 is also specific to each individual ear of the patient. This may provide a more comfortable fit compared to a one size fits all mold, allowing the patient to sleep more comfortably while using the wireless audio device. Interior portions of the earbud 160 and internal componentry 150 are described in greater detail below. Additionally or alternatively, mold 162 may be one of a plurality of templates, wherein the templates are configured to fit differently sized ears. For example, there may be three templates configured to fit small, medium, and large ears, wherein small is smaller than medium and medium is smaller than large.

Turning now to FIG. 1D, it shows an internal view of the right earbud 160. It will be appreciated that the right earbud 160 may be substantially similar to a left earbud. The right earbud 160 comprises a silicone molding 162 lacquered with a silicone lacquer. The earbud 160 further comprises one or more audio transducers 172, wherein a transducer of the transducers 172 is located between an earpiece 173 and a resistor 174. The earpiece 173 may be between 1-3 millimeters. In one example, the earpiece 173 is exactly two millimeters. The transducer cable 152 may be between 200-300 millimeters in length. In one example, the transducer cable 152 is exactly 240 millimeters in length. A resistor may be placed interior to the band (e.g., band 110 of FIGS. 1A and 1B).

The transducer cable 152 is one of two transducer cables, wherein there exists a transducer cable for each earbud. Specifically, a right transducer cable is coupled to a right earbud and right extreme end of the wireless audio device and a left transducer cable is coupled to a left earbud and left extreme end of the wireless audio device.

Turning now to FIG. 2, it shows a perspective view 200 of the device 100. However, in the perspective view 200, the device 100 further comprises right 160 and left 170 earbuds, a right transducer cable 212, a left transducer cable 214, and differently shaped first 102 and third 106 buttons. Additionally, the perspective view 200 further illustrates a button 204 located on the left extreme end 112, which may function similarly to the second button 104 as described above. As such, the second button 104 may adjust a volume output of the right earbud 160 and the button 204 may adjust a volume output of the left earbud 170. In this way, tinnitus may be accurately treated via adjustable sounds emitting from the right 160 and left 170 earbuds.

Turning now to FIG. 3, it shows a system 300 depicting hardware connectivity between the wireless audio device 100 and auxiliary devices. Components of the wireless audio device 100 are illustrated within the dashed box. A controller, a battery, a power management device, a Wi-Fi receiver and/or transmitter, a real-time clock, a flash memory, an audio amplifier, a user interface, and earbuds. The power management device includes a charge regulator, a coulomb counter, and main power regulators. The controller is coupled to each of the above described components except for the batter and the earbuds. The user interface may include one or more of the buttons described above, thereby allowing a user (e.g., a patient and/or health care provider) to modify controller operating parameters. As described above, the user interface buttons may allow the user (e.g., a patient) to adjust operation of the device 100. For example, the user may play and pause audio, adjust volume settings, and connect to Wi-Fi. The user interface for provides the user with the ability to turn off the device 100. In some examples, a standby feature may be incorporated. The controller may include instructions for executing the standby feature wherein the real-time clock tracks a duration of time that audio has been paused. If the duration of time is greater than a threshold pause duration (e.g., 15 minutes), then the controller may turn off the device without instructions from the user. The audio amplifier may receive instructions from the controller to adjust a volume output of the earbuds. The instructions may be based on prior therapy sessions, stored biometric data, and/or user inputs.

In some examples, the controller may include instructions for playing a voice through the earbuds alerting a user of a change in operating parameters. For example, the voice may be programmed to say, when appropriate, ‘Wi-Fi on’, ‘connected’, ‘Power off’, ‘battery low’, etc.

The real-time clock enables the controller to track a duration of an ongoing activity. For example, the real-time clock may allow the controller to track a duration of a sleep cycle, duration of a therapy session, and/or provide time stamps regarding changes in wireless audio device 100 activity. The flash memory enables the device to save biometric data and other portions of a therapy session for a threshold amount of time. For example, the threshold amount of time is 90 days. The data and other stored information may be erased from the flash memory following the earlier of the 90 day threshold being reached or the data being transmitted to an auxiliary device. This may ensure memory is available for future therapy sessions.

Data from the device 100 may be transmitted to auxiliary devices via Wi-Fi. The auxiliary devices may include a computer, cell phone, tablet, or other computing device capable of connecting to Wi-Fi and storing data. The auxiliary devices may belong to the healthcare provider or the patient. In some examples, data is sent to auxiliary devices belong to both the healthcare provider and the patient. In this way, both the health care provider and the patient may access the patient's therapy session data sets.

The connection between the device 100 and the auxiliary device may be mediated through a web application software. The software will be a “class A-no injury or damage to health is possible” form of software. The software is downloaded and/or installed onto personal computers, tablets, and/or mobile devices readily available to the health care provider and patient. Additionally or alternatively, the software may be accessed from personal computers without download. As such, the software may be accessed via the internet. The software includes a user interface, an HTML/Javascript, angular+libraries, and server API module. The user interface may further include modules on the software configured to allow the patient to review their treatment progress, communicate with their health care provider, and select different tinnitus sound matches. The application may provide an interface to allow the patient to monitor their treatment. Usage data includes treatment duration, when the treatment was played, any adjustments to the amplitude, and the battery state and the beginning and end of the therapy. The Patient App requires a login which is authenticated by the server. The patient logs in with a unique user ID and password. Once authenticated, the patient only has access to their own session data. All server functions will be accessed via the Server API Module.

The Patient App is a web based single page application using HTML and JavaScript in the browser and using the Server API to communicate with the back end. The Server API Module provides an encapsulation of the server functions in a convenient form. It provides a JavaScript API and communicates to the server via TLS using RESTful interface calls. Parameters are validated where possible. The HTML/JavaScript layer uses a number of components, such as AngularJS, and supporting components to provide a single page web application framework.

A health care provider (HCP) device uses a secure TLS connection to a server to provide, generate and refine therapies for a patient, and to provide information on therapy usage by the patient. All server functions will be accessed via a server API module. HCPs login with a unique user ID and password. The HCP can only access and modify information for their own patients. Sound match generation and control will be done through HTTP with encrypted payload requests to the Wireless Earbuds.

The Provider App is a web based single page application using HTML and JavaScript in the browser and using the Server API to communicate with the back end. The Server API Module provides an encapsulation of the server functions in a convenient form. It provides a JavaScript API and communicates to the server via TLS using RESTful interface calls. Parameters are validated where possible. The HTML/JavaScript layer uses a number of components, such as AngularJS, and supporting components to provide a single page web application framework. The Provider App may play a Sound Match for 5 minutes. This ensures that it is not used instead of the Patient App.

The device 100 further includes a sensor coupled to the controller. One or more sensors may be located in one or more of the earbuds and the band (e.g., band 110 of the wireless audio device 100 shown in FIG. 1A). The sensors are configured to monitor biometric data of the patient. As described above, the device 100 may be worn around a neck of the patient and rest atop the patient's shoulders. The earbuds are inserted into each of the patient's ears. As such, sensors located in the earbuds may gather different biometric data than sensors located in the band.

FIG. 4 shows an example method 400 for generating a tinnitus therapy using instructions stored on and executed by the controller of the wireless audio device, as explained with regard to FIGS. 1A-1D and FIG. 2. For example, the wireless audio device may include tinnitus sound templates, the tinnitus sound templates including tinnitus therapy sound types, in order to generate a tinnitus therapy sound (e.g., tinnitus sound match). As such, the wireless audio device may be used to generate a tinnitus therapy based on the selected tinnitus therapy sound templates and adjustments made to the selected tinnitus therapy sound templates and/or the tinnitus therapy sound.

The method 400 begins at 402 where a sound survey is displayed. The method at 402 may further include completing the sound survey. In one example, completing the sound survey may include receiving inputs via inputs (e.g., adjustment buttons) displayed on the user interface via the display screen. For example, the sound survey may include a hearing threshold data input and the selection of sound templates. In another example, the sound survey may include a hearing test. The hearing test may include generating an audiogram based on the hearing test data. The method at 402 for completing the sound survey is shown in further detail at FIGS. 5A-5B. In one example, the tinnitus sound templates may include two or more of a cricket noise sound template, a white noise sound template, a pink noise sound template, a pure tone sound template, a broad band noise sound template, an amplitude modulated sine wave template, and a combination pure tone and broad band noise sound template. In an additional example, the sound templates selected may be a combination of at least two tinnitus therapy sound templates.

At 404, the method includes determining if the tinnitus therapy sound template(s) have been selected. Once the template(s) are selected, at 406, a tinnitus therapy sound may be stored on the wireless audio device based on the sound survey and adjustments made to the frequency and intensity inputs. Herein, a tinnitus therapy sound may also be referred to as a tinnitus therapy sound match and/or tinnitus sound match. In one example, a single tinnitus therapy sound template may be selected and subsequently the tinnitus therapy sound template may be adjusted. Specifically, two tinnitus therapy sound templates may be selected. As such, a first tinnitus therapy sound template and a second tinnitus therapy sound template may be adjusted separately. For example, generating a tinnitus sound may include adjusting firstly a white noise sound template and secondly a pure tone sound template. In another example, a first tinnitus therapy sound template and a second tinnitus therapy sound template may be adjusted simultaneously. In this way, generating a tinnitus sound may include adjusting a white noise sound template and a pure tone sound template together. Once the adjustments to the tinnitus therapy sound template(s) are made, the tinnitus sound templates may be combined to make a specific tinnitus therapy sound. In one example, a generated tinnitus therapy sound may be played to a user to determine if the tinnitus therapy sound resembles the patient's perceived tinnitus. The generated tinnitus therapy sound may need additional adjustments and a first and/or second tinnitus therapy sound template may be re-adjusted. A tinnitus therapy sound may be generated following the additional adjustments of the tinnitus therapy sound template(s).

Further, generating a tinnitus therapy sound may, also include adjusting firstly a white noise sound template and secondly a broad band noise sound template. In an additional example, generating a tinnitus sound match may include adjusting firstly a pure tone sound template and secondly a broad band noise sound template. In another example, generating a tinnitus sound match may include adjusting firstly a cricket noise sound template and secondly a white noise sound template.

Additionally, generating a tinnitus sound match may include three or more tinnitus therapy sound templates. As such, a combined tinnitus therapy sound match may include, in one example, adjusting firstly a pure tone sound template, secondly a broad band noise sound template, and thirdly a white noise sound template. In another example, a combined tinnitus therapy sound match may include adjusting firstly a cricket noise sound template, secondly a broad band noise template, and thirdly a white noise sound template. In an additional example, a combined tinnitus therapy sound match may include adjusting firstly a white noise sound template, secondly a pure tone sound template, thirdly a broad band noise template, and fourthly a cricket noise sound template.

Further, therapy parameters may be added to the tinnitus therapy sound to finalize the tinnitus therapy sound. In one example, therapy parameters may include adding a help-to-sleep feature, setting the maximum duration of the tinnitus therapy, and allowing a user to adjust the volume during the tinnitus therapy. At 408, the tinnitus therapy sound may be saved and finalized. Once the tinnitus therapy sound is finalized, the tinnitus therapy is complete and may be sent to the patient's device. In one example, the healthcare professional's device is configured to hold instructions executable to send the generated tinnitus therapy sound to a second physical, non-transitory device (e.g. the patient's device). In another example, finalizing the tinnitus therapy sound includes assigning the generated tinnitus therapy sound to an individual patient of the individual patient audiogram. Assigning the tinnitus therapy sound also includes storing the generated tinnitus therapy sound with a code corresponding to the individual patient.

Now referring to FIGS. 5A-5C, an example method 500 for generating the sound survey, including adjusting tinnitus sound templates is shown. The sound survey may include inputting hearing threshold data determined by an audiogram and selecting tinnitus therapy sound templates in order to create a tinnitus therapy sound. As such, a tinnitus therapy sound template may be selected based on the similarity of the tinnitus therapy sound template (e.g. tinnitus sound type) to the patient's perceived tinnitus. The sound survey is an initial step in generating a tinnitus therapy sound such that the template(s) selected will be adjusted following the conclusion of the sound survey.

FIG. 5A shows example tinnitus therapy sound template selections including sound template adjustment parameters. Creating a tinnitus therapy may include presenting each of a white noise, a pink noise, a pure tone, a broad band noise, a combined pure tone and broad band noise, a cricket noise, and an amplitude modulated sine wave tinnitus therapy sound template to a user. In an alternate embodiment, creating a tinnitus therapy may include presenting a different combination of these sound templates to a user. For example, creating a tinnitus therapy may include presenting each of a white noise, a pink noise, a pure tone, a broad band noise, and a cricket noise tinnitus therapy sound template to a user. In yet another example, creating the tinnitus therapy may include presenting each of a white noise, a pure tone, and a combined tone tinnitus therapy sound template to a user. The combined tone may be a combination of at least two of the above listed sound templates. For example, the combined tone may include a combined pure tone and broad band noise tinnitus therapy sound template.

After playing each of the available tinnitus therapy sound templates, the user may select which sound type, or sound template, most resembled their perceived tinnitus. In this way, generating a tinnitus therapy sound may be based on the tinnitus therapy sound template selected by the user. After selecting one or more of the tinnitus therapy sound templates, the selected sound template(s) may be adjusted to more closely resemble the patient's perceived tinnitus. Adjusting the tinnitus therapy sound, or tinnitus therapy sound template, may be based on at least one of a frequency parameter and an intensity parameter selected by the user. As discussed above, a tinnitus therapy sound template(s) may be selected if the tinnitus therapy sound(s) resembles the perceived tinnitus sound of a patient. However, in one example, a patient's perceived tinnitus sound may not resemble any of the tinnitus therapy sound templates. As such, at 558, an unable to match input may be selected. Upon selection of an individual tinnitus therapy sound template, a tinnitus therapy sound template may include adjustment inputs including adjustments for frequency, intensity, timbre, Q factor, vibrato, reverberation, and/or white noise edge enhancement. The pre-determined order of adjustments of the tinnitus therapy sound template(s) selections are described below with regard to FIG. 5A.

FIG. 5A begins at 502, by selecting a white noise sound template. White noise sound template adjustments may include, at 504, adjustments for intensity and adjustments for reverberation, at 506. For example, adjusting the tinnitus therapy sound may be first based on the intensity parameter and second based on a reverb input when the tinnitus therapy sound template selected by the user is the white noise tinnitus therapy sound template. If a pink noise template is selected at 503, the pink noise sound template may be adjusted based on intensity at 505 and reverberation at 507. Adjustments to the pink noise sound template may be similar to adjustments to the white noise sound template. For example, adjusting the tinnitus therapy sound may be first based on the intensity parameter and second based on a reverb input when the tinnitus therapy sound template selected by the user is the pink noise tinnitus therapy sound template. In another example, a pure tone sound template, at 508, may be selected. A pure tone sound template may be adjusted based on frequency, at 510, and intensity, at 512. In addition, a pure tone sound template may be further adjusted base on timbre, at 514. In one example, timbre may include an adjustment of the harmonics of a tinnitus therapy sound including an octave and/or fifth harmonic adjustments. Further, a pure tone sound template may be adjusted based on a reverberation, at 516, and a white noise edge enhancement, at 518. In one example, adjusting the tinnitus therapy sound may be first based on the frequency parameter, second based on the intensity parameter, third based on one or more timbre inputs, further based on a reverberation (e.g., reverb) input, and fifth based on an edge enhancement input when the tinnitus therapy sound template selected by the user is the pure tone sound template. In another example, a white noise edge enhancement may be a pre-defined tinnitus therapy sound template. Herein, a white noise edge enhancement sound template may be referred to as a frequency windowed white noise sound template. Additionally, a white noise edge enhancement adjustment may include adjusting the frequency windowed white noise based on an intensity input.

Continuing with FIG. 5A, a broad band noise sound template, at 520, may be selected. A broad band noise sound template may include an adjustment for frequency, Q factor, and intensity, at 522, 524, and 526, respectively. Further adjustments to a broad band noise sound template may include reverberation, at 528, and white noise edge enhancement, at 530. For example, adjusting the tinnitus therapy sound may be first based on the frequency parameter, second based on a Q factor input, third based on the intensity parameter, fourth based on a reverberation input, and fifth based on an edge enhancement input when the tinnitus therapy sound template selected by the user is the broad band noise tinnitus therapy sound template.

At 532, a combination tinnitus sound template may be selected. A combination tinnitus sound template may include both a pure tone and a broad band noise sound. As such, the combination pure tone and broad band noise sound template may include adjustments for frequency, Q factor, and intensity, at 534, 536, and 538, respectively. A combination pure tone and broad band noise sound template may include further adjustments for timbre, reverberation, and white noise edge enhancement, at 540, 542, and 544, respectively. For example, adjusting the tinnitus therapy sound may be first based on the frequency parameter, second based on a Q factor input, third based on the intensity parameter, fourth based on a timbre input, fifth based on a reverberation input, and sixth based on an edge enhancement input when the tinnitus therapy sound template selected by the user is the combined pure tone and broad band noise tinnitus therapy sound template.

At 546, a cricket noise sound template may be selected. A cricket noise sound template may include adjustments for frequency, at 548, and intensity, at 550. Further adjustments to a cricket noise template may include a vibrato adjustment, at 552. A vibrato adjustment may include adjustment to the relative intensity of the cricket noise sound template. A cricket noise sound template may also include adjustments for reverberation, at 554, and white noise edge enhancement, at 556. For example, adjusting the tinnitus therapy sound may be first based on the frequency parameter, second based on the intensity parameter, third based on a vibrato input, fourth based on a reverberation input, and fifth based on an edge enhancement input then the tinnitus therapy sound template selected by the user is the cricket noise tinnitus therapy sound template.

At 555, an amplitude modulated sine wave sound template may be selected. In one example, the amplitude modulated sine wave template may include a base wave and carrier wave component. Additionally, the amplitude modulated sine wave template may include adjustments for intensity (e.g., amplitude) at 557, or alternatively adjustment to the base wave frequency. In alternate embodiments, additional or alternative adjustments may be made to the amplitude modulated sine wave sound template.

In another embodiment, the tinnitus therapy sound template(s) may include a plurality of tinnitus therapy sounds including but not limited to the tinnitus therapy sounds mentioned above with regard to FIG. 5A. For example, FIG. 5A may include alternative or additional sound templates which may be displayed and played for the user. Specifically, in one example, an additional combination tinnitus sound template may be presented to and possibly selected by the user. In one example, the additional combination tinnitus therapy sound template may include a combined white noise and broad band noise sound template. In another example, the additional combination tinnitus therapy sound template may include a template combining more than two tinnitus therapy sound types.

It should be appreciated that once a user selects a sound template and its properties (such as intensity or frequency), no additional modulation is applied to the selection. Further it should be appreciated that once a user selects a sound level, treatment or therapy where the selected sound is replayed occurs at the selected sound level without lowering.

Referring now to FIG. 5B, method 500 begins at 560 by obtaining audiogram data via an audiogram input and/or patient hearing data. The audiogram input may include hearing threshold data. In one example, the hearing threshold data may be determined at an earlier point in time during a patient audiogram. An individual patient's hearing threshold data may include decibel and frequency data. As such, the frequency, expressed in hertz (Hz), is the “pitch” of a sound where a high pitch sound corresponds to a high frequency sound wave and a low pitch sound corresponds to a low frequency sound wave. In addition, a decibel (dB) is a logarithmic unit that indicates the ratio of a physical quantity relative to an implied reference level such that the physical quantity is a sound pressure level. Therefore, the hearing threshold data is a measure of an individual patient's hearing level or intensity (dB) and frequency (Hz). Additionally, the audiogram input and/or patient hearing data may be received by various methods. Based on a generated audiogram from the hearing test, a user may input hearing level and frequency data when prompted by the user interface. In yet another example, the audiogram input of patient hearing data may be uploaded to the healthcare professional's device via a wireless network, a portable storage device, or another wired device. In another example, the audiogram or patient hearing data may be input by the user (e.g., medical provider) with the user interface of the healthcare professional's device.

At 562, the method includes determining if the hearing threshold data from the audiogram has been received. Once the audiogram data has been received, at 564, the initial tinnitus therapy sound template settings (e.g. frequency and intensity) may be modified by the hearing threshold data from an individual patient's audiogram. For example, in order for the tinnitus therapy sound template to be in the correct hearing range of an individual patient, specific frequency and intensity ranges may not be included in the tinnitus therapy sound template. Specifically, if an audiogram's hearing threshold data reflects mild hearing loss of a patient (e.g. 30 dB, 3000 Hz), the frequency and intensity range associated with normal hearing will be eliminated from the template default settings (e.g. 0-29 dB; 250-2000 Hz) such that a default setting starts at the hearing level of the patient. In one example, an audiogram may include a range of frequencies including frequencies at 125 Hz, 250 Hz, 500 Hz, 1000 Hz, 2000 Hz, 3000 Hz, 4000 Hz, 6000 Hz, 8000 Hz, 10,000 Hz, 12,000 Hz, 14,000 Hz, 15,000 Hz, and/or 16,000 Hz.

Additionally, the hearing threshold data from an individual patient's audiogram may be used to determine sensitivity thresholds (e.g. intensity and frequency) of the tinnitus therapy sound. For example, hearing threshold data may include maximum intensity and frequency thresholds for an individual patient such that the tinnitus therapy sound template's intensity and/or frequency may not be greater than a patient's sensitivity threshold. As such, the sensitivity levels will further limit the intensity and frequency range of the tinnitus therapy sound template. As such, the frequency and intensity range of the tinnitus therapy sound template may be based on the hearing level and hearing sensitivity of the patient. Therefore, at 564, the tinnitus therapy sound template(s) default settings are adjusted to reflect the audiogram, hearing threshold data, and hearing sensitivity of the patient.

At 566, a plurality of tinnitus therapy sound templates may be displayed. In one example, the tinnitus therapy sound templates may include tinnitus sounds including cricket noise, white noise, pink noise, pure tone, broad band noise, amplitude modulated sine wave sound, and a combination of pure tone and broad band noise. Specifically, each tinnitus therapy sound template may be pre-determined to include one of the above listed tinnitus sounds having pre-set or default sound characteristics or template settings (e.g., frequency, intensity, etc.). As described above, in other examples more or less than 6 different tinnitus therapy sound templates may be displayed.

At 568, the tinnitus therapy sound template selection process begins by playing pre-defined tinnitus therapy sounds (e.g., sound templates). In one example, the pre-defined tinnitus therapy sounds may be played in a pre-determined order including playing a white noise sound first followed by a pink noise sound, pure tone sound, a broad band sound, a combination pure tone and broad band sound, a cricket noise sound, and amplitude modulated sine wave sound. In another example, the tinnitus therapy sounds may be played in a different order. Further, the different tinnitus therapy sounds may either be presented/played sequentially (e.g., one after another), or at different times. For example, the sound templates may be grouped into sound categories (e.g., tonal or noise based) and the user may be prompted to first select between two sound templates (e.g., cricket and white noise). Based on the user's selection, another different pair of sound templates (or tinnitus therapy sounds) may be displayed and the user may be prompted to select between the two different sound templates. This process may continue until one or more of the tinnitus therapy sound templates are selected. In this way, the method 500 may narrow in on a patient's tinnitus sound match by determining the combination of sound templates included in the patient's perceived tinnitus sound.

FIG. 5D presents an example method 590 of an order of presenting the different tinnitus therapy sounds (e.g., sound templates) to the user. As such, method 590 may be performed during step 568 in method 500. At 592, the method includes presenting a user, via a user interface of the healthcare professional's device, with a noise-based sound template and a tone-based sound template. The noise-based sound template may be a white noise sound template, a broad band noise sound template, a pink noise sound template, or some combination template of the white noise, broad band noise, and/or pink noise sound templates. The tone-based sound template may be a pure tone sound template, a cricket sound template, or some combined pure tone and cricket sound template.

At 594, the method includes determining if the noise-based sound was predominantly selected. In one example, the noise-based sound may be predominantly selected if an input selection of the noise-based sound is received. In another example, the user interface of the healthcare professional's device may include a sliding bar between the noise-based and tone-based sounds. In this example, the noise-based sound may be predominantly selected if an input (e.g., a sliding bar input) is received indicating the tinnitus sound is more like the noise-based sound than the tone-based sound. If an input of a predominantly noise-based sound is received, the method continues on to 596 where the method includes presenting the user with a white noise sound, a pink noise sound, and/or a broad band noise sound. The method then returns 570 in FIG. 5B. In one example, a patient may be presented with two different noise based sounds and then be able to use a slide bar to select whether the tinnitus sound sounds more like a first sound or a second sound. It should be appreciated that the sound may be selected for the left or the right or both. Conversely at 594, if the noise-based sound is not predominantly selected, the method continues on to 598 to present the user with a pure tone sound and a cricket sound. The method then returns to 570 in FIG. 5B. Other methods of presenting the different sound types (e.g., templates) to a user are possible and may include presenting the sound templates in different combinations and/or orders.

Following the presentation of the tinnitus therapy sound template, the user interface of the healthcare professional's device will display a prompt to the user confirming the tinnitus therapy sound template selection. For example, confirming the tinnitus therapy sound template selection may include selecting whether the selected sound template is similar to the patient's perceived tinnitus. At 570, the method 500 includes determining if a white noise sound is selected. In one example, a white noise sound may be selected if the presented white noise sound resembles a patient's perceived tinnitus. At 570, if a white noise sound is selected as a tinnitus sound similar to that of the patient's, the method continues on to 572 to display a white noise sound template. In one example, upon selection of a tinnitus therapy sound template, a tinnitus sound, corresponding to the selection, will be presented to the user. Following the presentation of the tinnitus therapy sound template, a user interface will display a prompt to the user confirming the tinnitus therapy sound template selection (e.g. white noise sound template). Once the tinnitus therapy sound template is selected, the user interface will display the tinnitus therapy sound template on the tinnitus therapy sound screen.

Method 500 continues to 573 in FIG. 5C where the method includes determining if a pink noise sound template is selected. If a pink noise sound template is selected as a tinnitus sound similar to that of the patient's, the method continues to 575 to display a pink noise sound template. If pink noise is not selected, the method continues on to 574 where the method includes determining if a pure tone sound template is selected. If a pure tone sound template is selected as a tinnitus sound similar to that of the patient's, at 576, the pure tone sound template is displayed in the and further adjustment to the pure tone sound template may be made. If a pure tone sound is not selected, at 578, the method includes determining if a broad band noise sound is selected. If a broad band sound template is selected as a tinnitus sound similar to that of the patient's, at 580, the broad band noise sound template is displayed and further adjustment to the broad band noise sound template may be made.

If a broad band noise sound is not selected, at 582, the method includes determining if a combination of pure tone and broad band noise sound is selected. If a combination of pure tone and broad band noise sound template is selected as a tinnitus sound similar to that of the patient's, at 584, the combination pure tone and broad band noise sound template is displayed and further adjustment to the combination pure tone and broad band noise sound template may be made.

If a combination of pure tone and broad band noise sound is not selected, at 586, the method includes determining if a cricket noise sound is selected. In one example, the user interface of the healthcare professional's device will prompt a user to select a cricket noise sound template. If the cricket noise sound template is selected, at 588, a user interface will display a cricket noise sound template.

If the cricket noise sound template is not selected at 586, the method continues to 587 to determine if an amplitude modulated sine wave template is selected. If the amplitude modulated sound template is selected, at 589, a user interface will display the amplitude modulated sine wave template. A user may then adjust an intensity and/or additional sound parameters of the sine modulated sine wave template. After any user inputs or adjustments, the method may include finalizing the tinnitus therapy sound including the amplitude modulated sine wave template.

An individual patient's perceived tinnitus may incorporate a plurality of tinnitus sounds; therefore, the method 500 may be repeated until all required templates have been selected. For example, a patient's perceived tinnitus may have sound characteristics of a combination of tinnitus sounds including white noise and broad band noise, white noise and pure tone, or pure tone and broad band noise. In yet another example, the patient's perceived tinnitus may include sound characteristics of two or more tinnitus sounds including two or more of white noise, pink noise, broad band noise, pure tone, amplitude modulated sine wave, and cricket. Additionally, the tinnitus therapy sound generated based on the selected tinnitus therapy sound templates may contain different proportions of the selected sound templates. For example, a generated tinnitus therapy sound may contain both pure tone and cricket sound components, but the pure tone component may make up a larger amount (e.g., 70%) of the combined tinnitus therapy sound. As such, two or more tinnitus therapy sound templates may be selected during the template selection process. In one example, a first tinnitus therapy sound template may include a white noise sound and a second tinnitus therapy sound template selection may include a pure tone sound. In another example, a first tinnitus therapy sound template may include a broad band noise sound template and a second tinnitus therapy sound template may include a white noise sound template. In another example, the first tinnitus therapy sound template may include a pure tone sound and a second tinnitus therapy sound template may include a broad band noise sound. In another example, a first tinnitus therapy sound template may include a cricket noise sound and a second tinnitus therapy sound template may include a white noise sound template.

In an additional example, a first tinnitus therapy sound template may include a pure tone sound template, a second tinnitus therapy sound template may include a broad band noise sound template, and a third tinnitus therapy sound template may include a white noise sound template. In another example, a first tinnitus therapy sound template may include a cricket noise sound template, a second tinnitus therapy sound template may include a broad band noise template, and a third tinnitus therapy sound template may include a white noise sound template. In an additional example, a first tinnitus therapy sound template may include a white noise sound template, a second tinnitus sound template may include a pure tone sound template, a third tinnitus therapy sound template may include a broad band noise template, and a fourth tinnitus therapy sound template may include a cricket noise sound template. After receiving one or more tinnitus therapy template selections, the selected tinnitus therapy template(s) may then be individually or simultaneously adjusted, to create the tinnitus therapy sound.

Now referring to FIG. 6, an example method 600 for generating an audiogram is shown including performing a hearing test. A hearing test may be performed during a sound survey including the tinnitus therapy sound template selection process, as described above with reference to FIG. 5B-5D. Further, the hearing test data may be used to generate an audiogram. A patient's audiogram may be used to set the pre-defined frequency and intensity parameters of the tinnitus therapy sound template(s).

At 602, the method includes displaying a hearing test for a user. In one example, a hearing test may include a hearing level and intensity table. The hearing level and intensity table may include a plurality of inputs including hearing level or intensity inputs and frequency inputs. In another example, the hearing level and intensity table may include a range of frequencies and intensities. At 604, the method includes determining if a hearing level and frequency input selection has been received. If an input selection has not been received, the method continues to display the hearing test. However, if a frequency and intensity input has been received, at 606, the method includes playing a pre-determined sound based on an input selection. In one example, if a user selects a frequency input and an intensity input, a corresponding sound may be presented to the user. In another example, a user interface may prompt a user to confirm if the sound played is within a user's hearing range. The method, at 608, includes adjusting the hearing test based on user frequency and intensity input selection. In one example, a hearing level and intensity table may be adjusted to include a range of frequencies and intensities based on the user selection. For example, frequencies and intensities that are not in the range of the user's hearing levels might not be available for selection by the user.

At 610, the method includes determining if the adjustment of the hearing data is complete. If the adjustment is not complete, the method continues, at 608, until the adjustment to the hearing data is completed. The method, at 612, includes generating and displaying an audiogram based on the adjusted hearing data. In one example, based on the user selected inputs, an audiogram might be displayed. An audiogram may include the hearing level and frequency of a patient. In another example, the generated audiogram may be used in the tinnitus therapy sound template selection. Further, the audiogram data may be used to set the pre-defined frequency and intensity levels of the tinnitus therapy sound template, as described above with reference to FIGS. 5B-5D. Additionally, the audiogram data and/or hearing test results may be stored in the healthcare professional's device and accessed via a questionnaires screen of the healthcare professional's device, the questionnaires screen including a list of any completed hearing tests.

Now referring to FIG. 7, an example method 700 for playing the therapy sound template through a wireless audio device is shown. In one example, the one or more therapy sound templates are stored on a memory of the wireless audio device and the templates are played when proper operating conditions are reached.

At 702, the method 700 includes determining if the wireless audio device is on. In one example, this is determined by monitoring a state of charge of a battery. If the state of charge is decreasing, then the device is on. If the state of charge is constant, then the device is off If the device is off, then the method proceeds to 704 to maintain current operating parameters and does not play therapy sounds, music, or monitor patient biometric data.

If the device is on, then the method 700 proceeds to 706 to determine if the device is not being charged. The device is not being charged if a state of charge of the battery is not increasing. If the state of charge of the battery is increasing, then the device is being charged and the method 700 proceeds to 704 to maintain current operating parameters and does not paly therapy sounds. Additionally the device may not monitor biometric data while the device is being charged. However, in some examples, the device may play music and/or other sounds unrelated to therapy while the device is being charged. Alternatively, the device is disabled from performing auditory functions outside of pre-programmed responses stored therein (e.g., ‘device on’, ‘connected’, etc.) when the device is charging.

If the device is not being charged, then the method 700 proceeds to 708 to determine if the patient is sleeping. The patient may be sleeping if sensors in the earbuds of the wireless audio device measure one or more of an amount of movement by the patient being less than a threshold movement, a temperature of the patient being less than a threshold temperature, a heart rate of the patient being less than a threshold heart rate, and a time. The threshold movement is based on an amount of movement stored in a look-up table corresponding to an amount of movement during sleep. In one example, the amount of movement stored in the look-up table is based on only the patient's average amount of movement during sleep. This may be tracked by an accelerometer arranged in one or more of the earbuds and/or band. Alternatively, the amount of movement stored in the look-up table is based on an average taken across a variety of patient's. The threshold temperature may be based on a sleeping temperature of the patient. An IR temperature sensor located in one or more of the earbuds and band may measure the patient's body temperature. In one example, the temperature sensor is directed toward the patient's ear canal. In some examples, the sleeping temperature is slightly lower than a patient's temperature while being awake. Likewise, the threshold heart rate may be based on a patient's sleeping heart rate. In one example, the sleeping heart rate is slightly lower than a patient's heart rate while being awake. Similarly, the threshold blood pressure may be based on a patient's sleeping blood pressure. In one example, the sleeping blood pressure is slightly lower than a patient's blood pressure while being awake. Lastly, time may be used to determine if the patient is sleeping. For example, the controller may comprise instructions stored in memory to predict when a patient may be sleeping based on data stored in a look-up table. For example, if a patient routinely goes to bed between 2200-2300, then it may be determined that the patient is sleeping at 2330.

If the patient is not sleeping, then the method 700 proceeds to 710 to maintain current operating parameters and does not play therapy sounds. Alternatively, the device may play music or other sounds unassociated with tinnitus templates stored on the device, unless otherwise selected by the patient (e.g., user). In this way, the wireless audio device may also be used as headphones, wherein the device may connect to a mobile device, for example, and music stored thereon may be played via the wireless audio device.

If the patient is sleeping, the method proceeds to 712 to monitor patient biometric data. One or more sensors located in the earbuds of the wireless audio device may monitor patient biometric data. Blood pressure, heart rate, body temperature, etc. may be estimated via one or more sensors located in the earbuds. As an example, body temperature may be estimated via a temperature sensor (e.g., a thermometer). As another example, heart rate may be estimated by periodically shining a light on a blood vessel and monitoring either an amount of light absorbed or an amount of light deflected by the blood vessel. In one example, if the light is green light, then absorption is measured. In another example, if the light is red light, then deflection is measured.

At 714, the method 700 determines if a desired sleep cycle is ongoing. In one example the desired sleep cycle corresponds to a patient's sleep cycle where tinnitus may interrupt the patient's sleep. In one example, the desired sleep cycle is a REM sleep cycle. Biometric data may differ between sleep cycles. For example, during REM sleep cycles, a patient's body temperature may fall to a threshold temperature, a patient's breathing may become more variable and increase relative to non-REM sleep cycles, and increased heart rate and blood pressure relative to non-REM sleep cycles. In one example, the threshold temperature is a lower body temperature (e.g., 36° C.) less than an average human body temperature.

Additionally or alternatively, the sleep cycles of the patient may be determined initially by measuring biometric data and timed. An average duration of each of the sleep cycles may be determined over time. Thus, the patient's sleep cycle may be determined based on a time elapsed since the patient fell asleep.

In some examples, the method may determine if the desired sleep cycle is upcoming. This may be determined by a shift in biometric data gathered in real-time shifting from a current sleep cycle to the desired sleep cycle. If the sleep cycle is upcoming, then the method may adjust tinnitus making or treatment sounds to correspond to the upcoming sleep cycle. This may further include gradually increasing a sound of the tinnitus making or treatment sounds until the desired sleep cycle begins. In one example, gradually increased the sound includes increasing a volume of the tinnitus making or treatment sounds by 10% per minute until the desired sleep cycle begins.

If the desired sleep cycle is not ongoing, then the method proceeds to 716 to continue monitoring biometric data and does not play therapy sounds. In some examples, therapy sounds may played through an entire duration of a patient's sleep. However, a volume of the therapy sounds may be adjusted based on the determined sleep cycle. As an example, the volume of therapy sounds may be louder during REM sleep cycles than non-REM sleep cycles.

If the desired sleep cycle is ongoing, then the method proceeds to 718 to play the tinnitus making or treatment sounds based on a selection made by the health professional and/or patient. As described above, one or more templates may be selected and merged together to form a variety of tinnitus making or treatment sounds. The method may select one of a plurality of the pre-selected templates based on current biometric data. For example, if heart rate is elevated, then a broad band noise may be selected. Alternatively, if heart rate is decreased, then white noise may be selected. Different pre-selected templates may be merged and played to maintain biometric data at a level desired, wherein the level desired is based on a current sleep cycle.

At 720, the method includes determining if a desired sleep cycle is still ongoing. The desired sleep cycle is no longer ongoing if biometric data is altered. For example, the REM sleep cycle is no longer ongoing if body temperature increases to a temperature greater than or equal to the threshold temperature. If the desired sleep cycle is no longer ongoing, then the method 700 proceeds to 722 to disable the making or treatment sounds and continues to monitor biometric data. In some examples, additionally or alternatively, one or more of a volume of the sounds are decreased and a different template is selected in response to the sleep cycle changing to a different sleep cycle (e.g, REM to non-REM).

In some examples, additionally or alternatively, the method may determine if the desired sleep cycle is approaching its conclusion. If the desired sleep cycle is approaching its conclusion (e.g., next sleep cycle is less than 10 minutes away), then the method may include gradually decreasing tinnitus making or treatment sounds (e.g., by 10% per minute) until the next sleep cycle begins.

If the desired sleep cycle is still ongoing, then the method proceeds to 724 to continue playing sounds and monitoring biometric data. In one example, the making or treatment sounds are adjusted during the desired sleep cycle if an undesired biometric response occurs during the desired sleep cycle. For example, if the average REM cycle for a given patient lasts between 10-30 minutes and a temperature of the patient rises to a temperature greater than the threshold temperature seven minutes into the REM cycle, then the method may adjust tinnitus making or treatment sound settings to restore biometric data to desired levels. In one example, the volume of the making or treatment sounds is increased.

FIGS. 8A-8B show an example method 800 for recording and tracking patient data. Method 800 further includes presenting the tracked data to a user and adjusting the tinnitus therapy based on the tracked data. Once a tinnitus therapy sound match (e.g., tinnitus therapy sound) is generated and uploaded onto a patient's device, a patient may be instructed to use the patient's device when they sleep. In one example, the patient's device is configured with instructions stored thereon that execute one or more tinnitus therapy sounds based on biometric data monitored from a patient's ears. In addition, the patient's device may record all performed actions to the device during usage. In one example, the patient's device may also track intensity adjustments to the generated tinnitus therapy sound over time. In this way, a physician may review and track the recorded data, thereby determining the progress of the tinnitus therapy. In addition, the accumulation of an individual patient's tracked data may generate a medical record including a patient audiogram, the tinnitus therapy sound, and a patient adjusted tinnitus therapy sound. At any rate, the recorded data may further comprise associated time stamps along with corresponding biometric data and patient feedback, when applicable. For example, the patient may wake up due to undesired tinnitus sounds, which may prompt the patient to rate the tinnitus therapy sound less than satisfactory. The less than satisfactory review along with the time stamp and biometric data are stored together.

In some embodiments, the patient or user may have one or more tinnitus sound matches (e.g., one or more generated tinnitus therapy sounds). For example, more than one tinnitus sound match may be generated and assigned to a single patient. In another example, the patient may adjust (e.g., alter) their sound match using the process described above in FIGS. 5A-5D on a device at home (e.g., the same or similar to the healthcare professional's device). In this way, the patient may generate and/or modify their tinnitus sound match to create new sound matches different than their original sound match. The patient may then choose to play any of their generated tinnitus sound matches based on changes to their perceived tinnitus sound and/or based on an indication from a healthcare professional. For example, the patient may listen to different tinnitus sound matches on different nights or during different sessions.

At 802, the method includes determining if a therapy session has started. In one example, a therapy session may not begin until a start button input is selected on the patient's device (e.g. first button 102 shown in FIG. 1A). Alternatively, the therapy session may automatically begin without input from the patient so long as the wireless audio device is on. As described above, sensors in the earbuds may monitor biometric data and activate one or more tinnitus therapy sounds in response to the biometric data gathered. Once the therapy session has started, at 804, therapy data from the patient's device may be recorded for the duration of the therapy session. In one example, recorded data may include a patient's information, biometric data (e.g., heart rate, blood pressure, body temperature, movement, etc.), date of the therapy session, time of day the therapy session, and/or volume usage (e.g. changes in intensity). The recorded data may further include the specific tinnitus therapy sound match that was played during a session. This may include an identifier (e.g., match 1 or match 2) for each tinnitus sound match created by a user or patient. For example, upon creation of a tinnitus sound match, the sound match may be assigned a unique name and/or identifying information that identifies and differentiates the unique sound match from other sound matches for a same patient (or user). If a patient has more than one tinnitus sound match, the recorded data may include individual intensity changes for each sound match. In another example, the recorded volume usage may include changes in intensity to both right and left ear inputs. As such, a user may change the intensity of the tinnitus therapy sound match at the start of the therapy session as well as during the therapy session. In another example, the patient's device may be continuously playing the tinnitus therapy sound without breaks and tracking intensity changes to the continuously played tinnitus therapy sound over time.

At 806, the method includes determining if the therapy session has ended. For example, in order for a therapy session to end, a finish button input may be selected. Alternatively, the therapy session may end after a therapy duration has passed. If the session has not ended, recording of the therapy data may be continued. Once a finish input has been selected, at 808, the recorded therapy data may be saved and stored on the patient's device, at 810. Following the conclusion of a tinnitus therapy session, for example, a plurality of tinnitus therapy sessions may be played on a patient's device. Therefore, an accumulation of recorded data may be saved and stored on a patient's device. At 812, the recorded therapy data may be uploaded. In one example, the patient's device may receive a signal from a healthcare professional's device (e.g. tablet, desktop computer, etc.) to upload the recorded therapy data. As such, uploading the recorded data may occur wirelessly. In another example, the uploaded data may include date of the therapy session, time of day the therapy session was played, and changes in intensity (e.g. volume usage). In yet another example, therapy data may also include metadata from the patient's device. Further, at 814, the patient's identification information is uploaded to a healthcare professional's device. In one example, a plurality of recorded data may be uploaded to a healthcare professional's device. As such, a patient medical record (e.g., report) may be generated. In one example, generating a patient medical record may include a patient audiogram, the combined tinnitus therapy sound, and a patient adjusted tinnitus therapy sound.

Further, the uploaded recorded data may be stored and saved on a healthcare professional's device, thereby allowing a physician to track the recorded data over multiple therapy sessions. As such, tracking changes to the therapy session over a duration of time may determine patient progress to the tinnitus therapy. In one example, tracking changes of a patient's device may include remotely tracking intensity changes to the combined tinnitus therapy sound. In another example, tracking changes of a patient's device may include remotely transferring tracked changes to a secured data network.

The method continues to 816 in FIG. 8B where the method includes presenting the tracked therapy data to a user (e.g., a patient and/or healthcare professional). In one example, after a duration or a series of therapy sessions, a patient may have an appointment with a healthcare professional. Additionally or alternatively, a patient may view the tracked data on their own. In one example, presenting the tracked therapy data includes presenting each of a volume evolution (e.g., intensity changes) and usage data of the tinnitus sound match. The volume evolution may include changes in an overall, right ear, and/or left ear volume of the played tinnitus sound match (e.g., the volume of the sound match as listened to by the patient). Additionally, the volume evolution may be presented as volume changes over time or over a series of sessions. Usage data may include a frequency of use or frequency of listening to the sound match. For example, presenting the usage data may include one or more of presenting a total number of sessions, a date of a first session, a date of a last (e.g., most recent) session, an average session length, an average daily usage (e.g., in hours per day), and/or an average weekly usage (e.g., days per week). Presenting therapy data may also include presenting therapy details such as the tinnitus sound matches (e.g., all the different sound matches used by the patient) and prescribed therapy parameters such as the help-to-sleep option and allow to adjust volume option. In one example, the tracked therapy data may be presented to the user via a user interface of the healthcare professional's device. Tracking and viewing the changes made to the tinnitus therapy sound match over a duration of time may aid in determining patient progress with the tinnitus therapy.

Method 800 may further include, at 818, generating a report based on the tracked therapy data. In one example, the report may include a session report showing data for a particular (e.g., selected) session (e.g., one night of listening to the sound match). The evolution report may present patient details, a volume evolution for a series of sessions, as well as usage data and sound match details (e.g., sound match composition such as pure tone or combined white noise and pure tone sound) for each session in the series of sessions. In one example, a healthcare professional may generate the report during an appointment with the patient. In another example, a user (e.g., patient) may create the report after tracking one or more therapy sessions.

In some examples, the tracked therapy data may be used to make changes to the generated tinnitus therapy sound match. Thus, at 820, the method may include adjusting the tinnitus therapy based on the tracked and presented therapy data. In one example, a user may adjust a patient's tinnitus sound match and/or therapy parameters of the tinnitus sound match based on the tracked data. More specifically, as one example, adjusting the tinnitus therapy may include changing one or more sound parameters of the tinnitus sound match. For example, intensity, frequency, or other sound parameters of one or more sound templates included in the tinnitus sound match may be adjusted. In another example, a new template may be added to the tinnitus sound match or another sound template may be removed from the tinnitus sound match. In another example, a new tinnitus sound match may be created including a different sound template than the original sound match. In yet another example, the prescribed duration of therapy, the day/night option, the help-to-sleep option, or the allow volume change option may be changed based on the tracked data. In this way, a user may utilize tracked data to guide tinnitus therapy changes in order to better treat the patient. In some examples, adjusting the tinnitus therapy may follow similar methods to those presented in FIGS. 5A-5D, as described above. By tracking patient therapy data over time and subsequently presenting the tracked data to a user, changes to (or the evolution of) a patient's tinnitus may be identified. Further, by adjusting the patient's tinnitus therapy (including the tinnitus sound match) based on the tracked therapy data, a more effective tinnitus treatment may be prescribed to the patient. As a patient's tinnitus continues to evolve over time, the tinnitus therapy may be updated to match a patient's perceived tinnitus sound and further reduce the patient's tinnitus.

In alternate embodiments, the methods presented for generating a tinnitus therapy sound or match may also be used to generate a sound or match for therapy of other neurological disorders. For example, the generated audio sound may be at least partially used for treating neurological disorders such as dizziness, hyperacusis, misophonia, Meniere's disease, auditory neuropathy, autism, chronic pain, epilepsy, Parkinson's disease, and recovery from stroke. In this embodiment, sound templates may be adjusted based on patient data, the patient data being specific to the neurological disorder. In some examples, different combinations of the above described sound templates may be used to generate an audio sound or match for one of the neurological disorders.

The healthcare professional's device may allow a healthcare provider to manage one or more patients or users. For example, the healthcare professional's device may include one or more administrative or patient management screens (e.g., user interfaces or displays) that enabled the healthcare provider to select and then manage data of one or more patients. For example, a patient may be selected and statistics (e.g., tracked data) may be provided to show a patient's progress or data tracking for a single session or a plurality of sessions. Information regarding the patient or patient's tinnitus therapy may be inputted, tracked and in some examples linked with other records or databases, including but not limited to digital medical records.

Turning now to FIG. 9, it shows a method 900 for adjusting the tinnitus making or treatment sounds near the beginning and/or conclusion of a current sleep cycle. In one example, the method 900 may be used in conjunction with any of the methods described above.

The method 900 may begin at 902, where the method determines a current sleep cycle. Non-REM and REM sleep cycles have disparate biometric data sets, wherein biometric data may be gathered via sensors located in the earbuds of the sound device. For example, if a patient's body temperature is less than the threshold temperature, then REM sleep may be occurring. Other biometric data values may further be used to determine the sleep cycle, such as, blood pressure, heart rate, respiration, etc., as described above.

At 904, the method includes determining if the sleep cycle is nearing its conclusion. This may be determined by monitoring a variation in biometric data or via empirical data stored in a look-up table. For example, if the current sleep cycle is a non-REM sleep cycle, then the non-REM sleep cycle may be nearing its conclusion if biometric data gathered shift toward biometric data gathered in the REM sleep cycle. As an example, if the patient's body temperature is decreasing, but does not fall below the threshold temperature, then the patient is in a non-REM sleep cycle nearing its conclusion. Alternatively, the data stored in the look-up table may comprise information regarding an average duration of the non-REM and REM sleep cycles for a given patient. For example, the patient may average non-REM sleep cycles lasting 80-100 minutes and REM sleep cycles lasting 15-30 minutes. As such, the real-time clock in the sound device may monitor a time elapsed in each cycle. If the time elapsed is within a threshold percentage of the average duration (e.g., within 80%), then the sleep cycle may be nearing its conclusion.

If the sleep cycle is not nearing its conclusion, then the method proceeds to 906 to maintain current therapy setting. This may include maintaining a volume of the therapy sounds for making or treating tinnitus. This may further include maintaining a type of therapy sound being played.

If the sleep cycle is nearing its conclusion then the method proceeds to 908 to gradually decrease a volume of the current tinnitus making or treatment sounds. Gradually decreasing the volume may include decreasing the volume incrementally until such that the volume drops by a relatively equal amount each increment until the sleep cycle is concluded. For example, the volume decreases by 10% of its original volume each increment and the volume of the current tinnitus making or treatment sound reaches zero substantially simultaneously to a conclusion of the current sleep cycle being reached.

In some examples, additionally or alternatively, the tinnitus making or treatment sound of the next sleep cycle may be phased with and/or merged with the current tinnitus making or treatment sound as the current sleep cycle approaches its conclusion. In one example, the sound mixing of the two sounds may include balancing volumes of the two sounds, wherein the sounds are adjusted in tandem. For example, if the current tinnitus making or treatment sound is decreased to 70% its original volume (e.g., 100%), then the tinnitus making or treatment sound of the next sleep cycle is increased to 30% of the original volume of the current tinnitus making or treatment sound. Once the current sleep cycle is concluded, the two sounds may both be substantially 50% volume each.

At 910, the method 900 includes determining if the next sleep cycle has begun. This includes measuring sufficient changes in body temperature, blood pressure, heart rate, and respiration to match biometric data parameters of the next sleep cycle. For example, if the next sleep cycle is a non-REM sleep cycle, then the next sleep cycle (e.g., non-REM) has begun if at least the patient's body temperature is greater than the threshold temperature.

If the next sleep cycle has not begun, then the method proceeds to 912 to continue to gradually decrease the volume of the current tinnitus making or treatment sounds as described at 908.

If the next sleep cycle has begun, then the method proceeds to 914 to gradually increase a volume of tinnitus making or treatment sounds for the newly initiated cycle. Gradually increasing the tinnitus making or treatment sounds may include incrementally increase the tinnitus making or treatment sounds linearly. For example, the tinnitus making or treatment sounds are increased by 10% of a target volume for each increment.

In one example, the tinnitus making or treatment sound may be phased with a tinnitus making or treatment sound of the previous sleep cycle. For example, if the next sleep cycle begins with two tinnitus making or treatment sounds each at 50% volume of the target volume, the tinnitus making or treatment sound desired for the current sleep cycle may increase to 60% while the tinnitus making or treatment sound of the previous sleep cycle may decrease to 40%. This pattern may continue until the desired tinnitus making or treatment sound is 100% and the tinnitus making or treatment sound of the previous sleep cycle is 0%.

Turning now to FIG. 10, it shows a method 1000 for calibrating one or more sensors of the tinnitus making or treatment device. In one example, biometric data gathered from one or more sensors is compared to determine if a calibration of one or more sensors is desired. The method 1000 may be executed when the tinnitus making or treatment device is on, earbuds are inserted into the patient's ears, band is around the patient's neck, and the patient is sleeping.

The method 1000 may begin at 1002, where the method compares biometric data gathered from each sensor. As described above, the device may comprise one or more sensors located in the left earbud, right earbud, and/or band for measuring biometric data. The controller may comprise instructions for comparing biometric data from each sensor to a set of threshold values. For example, a body temperature measured by a sensor in the right earbud may be measured against a right earbud threshold temperature while a body temperature measured by a sensor in the band may be measured against a band threshold temperature.

At 1004, the method includes determining if calibration is needed. For example, if body temperatures measured at the left earbud and the band indicate a patient is in a REM sleep cycle, but a body temperature measured at the right earbud indicates a patient is in a non-REM sleep cycle, then the right earbud may desire calibration.

In some examples, additionally or alternatively, the method may further include comparing biometric data gathered from each of the sensors to data stored in a look-up table. For example, if data in the look-up table indicates the patient is in REM sleep and biometric data gathered from the left earbud and band indicate the patient is in REM sleep, but the right earbud indicates the patient is in non-REM sleep, then the right earbud may need calibration.

If calibration is not needed, and each of the sensors located in one or more of the left and right earbuds and neck are operating as desired, then the method 1000 proceeds to 1006 to maintain current operating parameters and the sensors are not calibrated.

If calibration is needed, then the method 1000 proceeds to 1008 to calibrate one or more sensors. Calibrating a sensor may include adjusting one or more algorithms and filters applied to raw biometric data gathered from the sensor according to differences determined above. For example, if the right earbud demands calibration, then the calibration may include adjusting data received from the right earbud to resemble data gathered from the left earbud. In some examples, additionally or alternatively, a flag may be included with information uploaded from the device to the healthcare provider and/or to the patient, wherein the flag indicates degradation of one or more sensors of the device.

Turning now to FIG. 11, it shows a method 1100 for determining a sleeping position of the patient. As such, the method 1100 is executed when the patient is sleeping and the tinnitus making or treatment device is on.

The method 1100 begins at 1102 where the method includes determining a current cycle. As described above, this may be based on biometric data gathered from one or more sensors located in the earbuds and band of the tinnitus making or treatment device.

At 1104, the method includes determining if the patient is sleeping on their left side. This may include a left ear of the patient being pressed against a surface (e.g., a pillow). The patient may be sleeping on their left side if a pressure measured by a sensor in the left ear bud is greater than a first threshold pressure. In one example, the first threshold pressure is based on a pressure induced onto the sensor when the left earbud is inserted into the patient's ear and the patient is standing. As such, if the patient is laying on their left side, a pressure imparted onto the sensor in the left ear may be greater than the first threshold pressure.

In some examples, additionally or alternatively, the method may determine that the patient is sleeping on their left side if a temperature measured by a sensor in the left earbud is greater than a first upper threshold temperature. In one example, the first upper threshold temperature is based on a temperature measurement when the patient's ear is pressed against a surface. As such, the first upper threshold temperature may be greater than a patient's body temperature (e.g., 37° C.). Additionally or alternatively, the first upper threshold temperature may be adjusted based on a current sleep cycle. For example, the first upper threshold temperature may be lower during REM sleep compared to non-REM sleep. Specifically, the first upper threshold temperature may be equal to a temperature within 36-38° C. when the current sleep cycle is a REM sleep cycle. Additionally, the first upper threshold temperature may be equal to a temperature within 38-40° C. when the current sleep cycle is a non-REM sleep cycle.

If the patient is not sleeping on their left side and the sensor measures a temperature less than the first upper threshold or a pressure less than the threshold pressure, then the method proceeds to 1106 to determine if the patient is sleeping on their right side. If a pressure imparted onto a sensor in the right earbud is greater than a second threshold pressure, then the patient may be sleeping on their right side. In one example, the second threshold pressure is substantially equal to the first threshold pressure. Additionally or alternatively, a temperature measured by the sensor in the right earbud may be compared to a second upper threshold temperature. If the temperature measured by the sensor is greater than the second upper threshold temperature, then the patient may be sleeping on their right side. In one example, the second upper threshold temperature is substantially equal to the first upper threshold temperature.

If the patient is not sleeping on their right side and a pressure is less than the second threshold pressure or a temperature is less than the second upper threshold temperature, then the method proceeds to 1108 to determine if a patient is sleeping on their back. The patient may be sleeping on their back if a pressure induced onto one or more sensors located in the band is greater than a third threshold pressure. The third threshold pressure is based on a pressure imparted onto one or more sensors of the neck band when the neck band is pressed against a surface of the patient's neck and another surface which the patient's neck rests against. In one example, the third threshold pressure is substantially equal to the first and/or second threshold pressures.

In some examples, additionally or alternatively, the patient may be sleeping on their back if a temperature measured by one or more sensor in the band of the device is greater than a third upper threshold temperature, wherein the third upper threshold temperature is based on a temperature measured when the patient's neck is pressed against a surface (e.g., a pillow). In one example, the third upper threshold temperature is substantially equal to the first and/or second upper threshold temperatures. In another example, the third upper threshold temperature is less than the first and/or second upper threshold temperatures. This may be due to greater air flow through a neck region of the patient compared to adjacent the patient's ears. Said another way, greater heat transfer may occur between an ambient atmosphere and the patient's neck compared to heat transfer at the patient's ears. As such, temperature measurements at the patient's neck may be lower than temperature measurements at the patient's ears. Furthermore, the third upper threshold temperature may be adjusted dependent on the current sleep cycle. For example, the third upper threshold temperature increases when the current sleep cycle is a non-REM sleep cycle compared to a REM sleep cycle. In one example, the third upper threshold temperature is equal to a temperature between 37-39° C. during the non-REM sleep cycle. Furthermore, the third upper threshold temperature is equal to a temperature between 35-37° C. during the REM sleep cycle.

If the patient is not sleeping on their back and a pressure measured by a sensor in the band is less than the third threshold pressure or a temperature measured by a sensor in the band is less than the third upper threshold temperature, then the method proceeds to maintain current operating parameters and does not adjust sensor feedback. In this way, each sensor of the earbuds and the band provides unadulterated feedback and biometric therefrom is not adjusted.

Returning to 1104, if the patient is sleeping on their left side and a pressure is greater than a first threshold pressure or a temperature is greater than a first upper threshold temperature, then the method proceeds to 1112 to adjust sensor feedback from the left earbud. Adjustments may include modifying one or more filters and/or algorithms. For example, a temperature received from the left earbud may be modified by a filter to a lower temperature to more accurately reflect a patient's current body temperature. As described above, the left earbud may measure a body temperature higher than an actual body temperature when the patient is laying on their left side with their left ear pressed against a surface. By adjusting sensor feedback, the body temperature measured may be closer to or substantially equal to the patient's actual body temperature, thereby providing more accurate biometric data to the patient and their healthcare provider. In one example, feedback from the left earbud is adjusted to resemble feedback from the right earbud. Alternatively, the adjustments are based on data stored in a multi-input look-up table. Data from the look-up table is gathered based on feedback from the right earbud and band, which are operating as desired. As such, feedback from the left sensor is adjusted to resemble the data gathered from the look-up table. Additionally or alternatively, feedback from one or more sensors in the left earbud may be ignored while the patient is laying on their left side.

Returning to 1106, if the patient is laying on their right side and a pressure is greater than a second threshold pressure or a temperature is greater than the second upper threshold temperature, then the method proceeds to 1114 to adjust sensor feedback from the right earbud. Temperature measurements from the right earbud are higher than an actual patient body temperature due to diminished thermal communication between the right ear and the ambient atmosphere. As such, feedback from the sensor is adjusted based on the increased temperature measurements to more accurately reflect the patient's actual body temperature. In one example, this may include adjusting feedback from the right earbud to resemble feedback from the left earbud. Alternatively or additionally, feedback from the right earbud may be ignored. In some examples, feedback from the right earbud may be adjusted based on data stored in a multi-input look-up table. For example, the data in the look-up table may comprise data sets corresponding to feedback from the earbuds and band. As such, feedback from the right earbud may be compared to a data set obtained from the look-up table corresponding to feedback from the left earbud and the band. The feedback from the right earbud may be adjusted to resemble biometric data gathered from the look-up table.

Returning to 1108, if the patient is sleeping on their back and a pressure is greater than a third threshold pressure or a temperature is greater than the third upper threshold temperature, then the method proceeds to 1116 to adjust sensor feedback from the band. One or more biometric measurements from the band may be unequal to actual biometric values of the patient. In one example, the feedback from the band is compared to values stored in a multi-input look-up table, wherein the values are obtained based on stored band values corresponding to a current feedback from the earbuds. As such, if current feedback from the band is unequal to values stored in the look-up table, then feedback from the band may be adjusted to resemble values in the look-up table. Additionally or alternatively, feedback from the band may be ignored when the patient is sleeping on their back.

In this way, a wireless audio device is configured to play tinnitus making or treatment sounds while a patient is sleeping. The patient may pre-select one or more individual and combined therapy sounds that sufficiently mask and/or obstruct the patient's tinnitus, wherein these preselected sounds are stored onto the wireless audio device. The wireless audio device further comprises one or more sensors for monitoring biometric data of a patient, wherein the sensors are pressed into the patient's ear. The device further comprises instructions for modifying the therapy by adjusting one or more of a volume and type of sound emitted from the wireless audio device. The technical effect of administrating tinnitus therapy based on biometric data gathered in real-time is to provide the patient with increased tinnitus masking. By doing this, the patient and healthcare provider may realize an enhanced tinnitus therapy customized for the patient based on feedback from the patient along with review and analysis of biometric data in response to the types of sounds administered in therapy.

1. A method, comprising:
gathering biometric data from one or more sensors located in an earbud of a wireless audio device, wherein the earbuds are pressed into a patient's ear, and where the wireless audio device is configured to analyze the biometric data and determine a current sleep cycle and administer a tinnitus sound therapy based on the current sleep cycle.

2. The method of claim 1, wherein administering the tinnitus sound therapy to a user's ear is adjusted responsive to biometric data of the user gathered during sleep.

3. The method of claim 1, wherein adjusting a selected sound of the current tinnitus therapy in response to transitions in a sleep cycle as sensed by the one or more sensors in real-time.

4. The method of claim 1, further comprising adjusting a volume of playing sounds through the earbuds in response to a sleep cycle, wherein the adjusting includes phase-in and phase-out volume timing.

5. (canceled)

6. The method of claim 1, wherein gathering the biometric data further comprises obtaining audiogram data, the audiogram data comprising decibel and frequency data.

7. The method of claim 6, further comprising producing the audiogram data via a user inputting a hearing level and frequency data when prompted by a user interface during a hearing test.

8. The method of claim 1, wherein determining the current sleep cycle comprises monitoring a patient's body temperature, blood pressure, heart rate, and respiration and determining if the current sleep cycle is REM or non-REM.

9. The method of claim 8, wherein the current sleep cycle is REM if the patient's body temperature is less than a threshold temperature.

10. The method of claim 8, further comprising decreasing a volume of the tinnitus sound therapy, which is a first tinnitus sound therapy, in response to the current sleep cycle nearing a conclusion.

11. The method of claim 10, further comprising increase a volume of a second tinnitus sound therapy, different than the first tinnitus sound therapy, in response to a next sleep cycle beginning.

12. A system, comprising:
a wireless audio device comprising a left earbud and a right earbud coupled to different extreme ends of a neckband;
a plurality of biometric sensors arranged in the left earbud, right earbud, and neckband is configured to sense a body temperature, a heart rate, a respiration, and a blood pressure of a patient on which the wireless audio device is arranged; and
a controller with computer-readable instructions stored on non-transitory memory thereof that when executed enable the controller to:
adjust a volume of a first cycle of a current tinnitus sound therapy in response to a current sleep cycle ending; and
adjust a volume of a second cycle of the current tinnitus sound therapy in response to a next sleep cycle beginning, wherein the second cycle comprises a tinnitus sound therapy different than that of the first cycle.

13. The system of claim 12, wherein the tinnitus sound therapy is generated via a user selecting one or more sound templates via a user interface as the left earbud and the right earbud play one or more sound templates directly to a user's ears.

14. The system of claim 13, wherein the one or more sound templates comprise a white noise, a pink noise, a pure tone, a broad band noise, a combined pure tone and broad band noise, a cricket noise, and an amplitude modulated sine wave.

15. The system of claim 12, wherein the instructions enable the controller to decrease the volume of the first cycle in response to the current sleep cycle ending, wherein the volume of the first cycle is gradually decreased.

16. The system of claim 12, wherein a therapy session, including the first cycle and second cycle, is saved locally on a flash memory of the wireless audio device, and where the therapy session is uploaded to a health care provider device via Wi-Fi.

17. The system of claim 16, wherein the therapy session comprises therapy data including a date, a time, and usage and intensity changes, wherein the therapy session is uploaded with a patient identification.

18. A method, comprising:
determining a sleep cycle in response to biometric data gathered from sensors located in one or more earbuds and playing sounds through the earbuds based on the sleep cycle.

19. The method of claim 18, wherein playing sounds includes playing a tinnitus sound match, wherein the tinnitus sound match comprises three of more sound templates, wherein sound templates include one or more of a white noise, a pink noise, a pure tone, a broad band noise, a combined pure tone and broad band noise, a cricket noise, and an amplitude modulated sine wave.

20. The method of claim 18, wherein playing sounds further comprises modifying a frequency and intensity of sounds based on a hearing threshold data gathered during an audiogram.

21. The method of claim 20, wherein the audiogram comprises receiving patient inputs regarding the hearing threshold data which includes a user hearing level and frequency.
